(19) United States
(12) Patent Application Publication
(10) Pub. No.: US 2024/0018248 A1
(43) Pub. Date: Jan. 18, 2024

(54) AN LTBR AGONIST IN COMBINATION THERAPY AGAINST CANCER

(71) Applicants: VIB VZW, Gent (BE); KATHOLIEKE UNIVERSITEIT LEUVEN, K.U. LEUVEN R&D, Leuven (BE); VRIJE UNIVERSITEIT BRUSSEL, Brussel (BE)

(72) Inventors: Gabriele Bergers, Gent (BE); Elizabeth Allen, Leuven (BE); Bruno Dombrecht, Gent (BE); Pascal Merchiers, Leuven (BE); Jo Van Ginderachter, Ninove (BE)

(21) Appl. No.: 18/255,433

(22) PCT Filed: Nov. 30, 2021
(86) PCT No.: PCT/EP2021/083595

§ 371 Date: Jun. 1, 2023

(30) Foreign Application Priority Data
Dec. 2, 2020 (EP) 20211335.3
Apr. 2, 2021 (EP) 21166846.2

(51) Int. Cl. C07K 16/28; A61P 35/00

(52) U.S. Cl. CPC C07K 16/2866; A61P 35/00; C07K 16/2878; C07K 16/2818; C07K 2317/569; C07K 2317/76; C07K 2317/92; C07K 2317/732; C07K 2317/35; A61K 2039/507

(57) ABSTRACT

The present invention relates to a combination comprising a Treg depletor and an LTBR agonist. Such a combination is particularly useful for use in the treatment of a cancer.

### CROSS-REFERENCE TO RELATED APPLICATIONS

This application is a national phase entry under 35 U.S.C. § 371 of International Patent Application PCT/EP2021/083595, filed Nov. 30, 2021, designating the United States of America and published in English as International Patent Publication WO 2022/117572 on Jun. 9, 2021, which claims the benefit under Article 8 of the Patent Cooperation Treaty to European Patent Application Serial No. 20211335.3, filed Dec. 2, 2020, and claims the benefit to European Patent Application Serial No. 21166846.2, filed April 2, 2021, the entireties of which are hereby incorporated by reference.

### FIELD OF THE INVENTION

The present invention relates to a combination comprising a Lymphotoxin Beta Receptor (LTBR) agonist and a regulatory T cell (Treg) depletor, and a composition comprising such a combination. The present invention is particularly useful as a combined therapy in the treatment of a cancer.

### BACKGROUND OF THE INVENTION

Treg cells are one of the integral components of the adaptive immune system whereby they contribute to maintaining tolerance to self-antigens and preventing auto-immune diseases. However, Treg cells are also found to be highly enriched in the tumour microenvironment of many different cancers. In the tumour microenvironment, Treg cells contribute to immune escape by reducing tumour-associated antigen (TAA)-specific T-cell immunity, thereby preventing effective anti-tumour activity. High tumour infiltration by Treg cells is hence often associated with an invasive phenotype and poor prognosis in cancer patients.

Acknowledging the significance of tumour-infiltrating Treg cells and their potential role in inhibiting anti-tumour immunity, multiple strategies have been proposed to modulate Treg cells in the tumour microenvironment. Several studies have demonstrated that depleting Tregs enhances tumor immunity and offers significant therapeutic benefit (see e.g. Tanaka and Sakaguchi 2019, Eur J Immuno 49:1140-1146).

For antibody-mediated killing of tumor Treg cells, surface molecules that are expressed on tumor-infiltrating Treg cells are good targets, especially if these are expressed specifically or at a much higher level on tumor-infiltrating Treg cells in comparison to other T cells. For example, the CC chemokine receptor 4 (CCR4) is highly expressed on suppressive Treg cells. Mogamulizumab (KW-0761) is an anti-CCR4 antibody with an afucosylated Fc region to increase antibody-dependent cellular cytotoxicity (ADCC). Through binding of CCR4 on Tregs and its ADCC activity, mogamulizumab is able to deplete FoxP3⁺ CD4 Tregs (Kurose et al. 2015, J Thorac Oncol 10:74-83 and Sugiyama et al. 2013, PNAS 110:17945-17650). Mogamulizumab has been approved in Japan and the US and several clinical trials are ongoing with mogamulizumab in monotherapy or in combination with anti-PD-1 or anti-PD-L1 antibodies.

CD25 is a key surface characteristic of Treg cell-function and its expression is controlled by Foxp3. Tumor-infiltrating Treg cells in mice and humans highly express CD25. It has been demonstrated that anti-CD25 antibodies with enhanced ADCC activity effectively depletes intra-tumoral Treg cells, increases effector to Treg cell rations and improves control over established tumors (Vargas et al. 2017, Immunity 46:577-586). The same authors also observed that Treg depletion with anti-CD25 antibody synergized with PD-1 blockade.

As another example, the G protein-coupled CC chemokine receptor protein CCR8 (CKRL1/CMKBR8/CMKBRL2) and its natural ligand CCL1 have been known to be implicated in cancer and specifically in T-cell modulation in the tumour environment. Eruslanov et al. (Clin Cancer Res 2013, 17:1670-80) showed upregulation of CCR8 expression in human cancer tissues and demonstrated that primary human tumours produce substantial amounts of the natural CCR8 ligand CCL1. This indicates that CCL1/CCR8 axis contributes to immune evasion and suggest that blockade of CCR8 signals is an attractive strategy for cancer treatment. Hoelzinger et al. (J Immunol 2010, 184:8633-42) similarly show that blockade of CCL1 inhibits Treg suppressive function and enhances tumour immunity without affecting Treg responses. Wang et al. (PIoSONE 2012, e30793) reported increased expression of CCR8 on tumour-infiltrating FoxP3+ T-cells and suggested that blocking CCR8 may lead to the inhibition of migration of Tregs into the tumours. Due to the high and relatively specific expression of CCR8 on tumour-infiltrating Tregs, monoclonal antibodies against CCR8 have been used for the modulation and depletion of this Treg population in the treatment of cancer (e.g. WO2018112032 A1 and WO2019/157098 A1). WO2018/181425 A1 showed that depletion of Tregs with an anti-CCR8 mAb is able to enhance tumour immunity. The effects are increased by combining Treg depletion with anti-CCR8 antibodies with anti-PD-1 antibody therapy, which even protected mice from a re-challenge with the same tumor type (WO2018/181425 A1). Through their neutralizing activity, these antibodies inhibit Treg migration into the tumour, reverse the suppressive function of Tregs and deplete intratumoural Tregs (WO2019/157098 A1). Recently, Wang et al. (Cancer Immunol Immonother 2020, https://doi.org/10.1007/s00262-020-02583-y) showed that CCR8 blockade could destabilize intratumoural Tregs into a fragile phenotype accompanied with reactivation of the antitumour immunity and augment anti-PD-1 therapeutic benefits.

CTLA-4 is a protein receptor that functions as an immune checkpoint. An important function of CTLA-4 is the down-regulation of CD80/86 expression in antigen-presenting cells, thereby inhibiting the activation of conventional T cells. While CTLA-4 is constitutively expressed on naïve Tregs, its expression is upregulated in tumor-infiltrating Treg cells. Blockade of the inhibitory activity of CTLA-4 on both effector and Treg cells results in enhanced antitumor effector T cell activity capable of inducing tumor regression. It has been suggested that the activity of anti-CLTA-4 antibody on the Treg cell compartment is mediated via selective depletion of tumor-infiltrating Treg cells, requiring Fc gamma receptor-expressing macrophages (Simpson et al. 2013, J Exp Med 210:1695-1710) and enhanced ADCC activity enhances anti-tumor response (Selby et al. 2013, Cancer Immunol Res 1:32-42).

CD38 is expressed by a population of Tregs that is more immunosuppressive than CD38-negative Tregs. Treatment of patients with an anti-CD38 antibody having ADCC, CDC and ADCP activity depleted CD38-positive immunosuppressive Treg cells (Krejcik et al. 2016, Bood 128:384-394).

TIGIT is a coinhibitory receptor on Tregs that promotes Treg suppressor function. Anti-TIGIT antibodies with ADCC activity have been shown to preferentially deplete Tregs and induce antitumor efficacy in monotherapy and in combination with an anti-PD-1 (Leroy et al. 2018, Cancer Res 78(13 Suppl) Abstract LB-114).

ICOS expression on Tregs is higher in the tumor microenvironment than in the blood or spleen, indicating its usefulness for preferential intra-tumoral Treg depletion, which was confirmed in mouse tumors (Sainson et al. 2019, https://doi.org/10.1101/771493). Anti-ICOS antibodies with ADCC activity, such as MEDI-570 and KY1044 are currently tested in a clinical trials in monotherapy or combination therapy with an anti-PD-L1 antibody.

OX-40, 4-1BB and GITR are members of the TNF receptor superfamily and are constitutively expressed by Treg cells and up-regulated upon T-cell receptor stimulation whereas they are induced in conventional T cells only after T-cell receptor stimulation. Treg depletion by anti-OX-40 antibodies via activating Fc gamma receptors has for example been shown by Bulliard et al. (2014, Immunol Cell Biol 92:475-80).

While the depletion of tumor-infiltrating Treg cells in cancer therapy has shown anti-tumor efficacy in preclinical and clinical studies, further improvements are still needed in relation to therapeutic efficacy and duration.

### SUMMARY OF THE INVENTION

The inventors have now surprisingly found that a combination comprising a Treg depletor and an LTBR agonist as detailed in the claims fulfils the above-mentioned need. In particular, the inventors have surprisingly found that a synergistic effect is observed when the Treg depletor and the LTBR agonist as defined in the combination of the present invention are used. The combination of the present invention therefore provide an improved tumour therapy.

It is thus an object of the invention to provide a combination comprising a Teg depletor and an LTBR agonist.

In a preferred embodiment, the Treg depletor binds to a cell surface marker of a Treg and has cytotoxic activity.

Preferably, the cell surface marker of a Treg is selected from the group consisting of CCR8, CCR4, CTLA4, CD25, TIGIT, OX40, ICOS, CD38, GITR, 4-1BB, NRP1 and LAG-3.

In a particular embodiment, the cell surface marker of a Treg is selected from CCR8, CLTA4, CCR4, CD25, TIGIT, and ICOS; preferably CCR8, CLTA4, CD25, and CCR4; most preferably CCR8 or CTLA4. In another particular embodiment, the cell surface marker of a Treg is selected from CCR8, CCR4, CD25, TIGIT, and ICOS; preferably CCR8, CD25, and CCR4; most preferably CCR8.

In another preferred embodiment, the cytotoxic activity of the Treg depletor is caused by the presence of a cytotoxic moiety that induces antibody-dependent cellular cytotoxicity (ADCC), induces complement-dependent cytotoxicity (CDC), induces antibody-dependent cellular phagocytosis (ADCP), binds to and activates T-cells, or comprises a cytotoxic payload.

In a particular embodiment of the present invention, the cytotoxic moiety comprises a fragment crystallisable (Fc) region moiety, in particular an Fc region moiety has been engineered to increase ADCC, CDC, and/or ADCP activity, such as through afucosylation or by comprising an ADCC, CDC and/or ADCP-increasing mutation.

In yet a further embodiment, the Treg depletor is an antibody that binds a cell surface marker of a Treg and has ADCC, CDC or ADCP activity. In a further embodiment, the Treg depletor is a CCR8 binding antibody having ADCC, CDC or ADCP activity.

In another particular embodiment of the invention, the Treg depletor comprises (a) an Fc region moiety that has ADCC, CDC and/or ADCP activity, and (b) at least one single domain antibody moiety that binds to a cell surface marker of a Treg.

In another particular embodiment, the Treg depletor is a non-blocking binder of a cell surface marker of a Treg.

Another object of the invention is to provide a composition comprising the combination of the present invention.

Yet another object of the present invention is to provide a bispecific molecule comprising an LTBR agonistic moiety and a Treg depleting moiety, wherein the bispecific molecule has cytotoxic activity, as well as a nucleic acid encoding such.

A further object of the present invention is to provide a combination comprising a Treg depletor and an LTBR agonist, a composition comprising such a combination, and a bispecific molecule comprising an LTBR agonistic moiety and a Treg depleting moiety, wherein the bispecific molecule has cytotoxic activity, for use as a medicine.

Another object of the present invention is to provide a combination comprising a Treg depletor and an LTBR agonist, a composition comprising such a combination, and a bispecific molecule comprising a Treg depleting moiety and an LTBR agonistic moiety, wherein the bispecific molecule has cytotoxic activity, for use in the treatment of a cancer. Preferably, the cancer is selected from the group consisting of breast cancer, uterine corpus cancer, lung cancer, stomach cancer, head and neck squamous cell carcinoma, skin cancer, colorectal cancer, and kidney cancer.

Yet another object of the present invention is to provide an LTBR agonist for use in the treatment of a cancer, wherein the treatment further comprises Treg cell depletion therapy.

In a particular embodiment, the LTBR agonist is an LTBR agonistic antibody; and the Treg cell depletion therapy comprises the administration of a CCR8 binding antibody having ADCC, CDC and/or ADCP activity.

A further object of the present invention is a Treg depletor for use in the treatment of a cancer, wherein the treatment further comprises the administration of an LTBR agonist.

In addition to the Treg depletor and the LTBR agonist, the therapy may comprise a further active ingredient. In a further embodiment, the further active ingredient is a checkpoint inhibitor. A checkpoint inhibitor is a compound that blocks checkpoint proteins from binding to their partner proteins thereby activating the immune system function. Preferably the checkpoint inhibitor blocks proteins selected from the group consisting of PD-1, PD-L1, B7-1 and B7-2. More preferably the checkpoint inhibitor blocks PD-1 or PD-L1. Preferred examples include anti-PD-1 and anti-PD-L1 antibodies. Preferred immune checkpoint inhibitors for use in the present invention are selected from nivolumab, pembrolizumab, atezolizumab, avelumab, durvalumab, cemiplimab, JTX-4014, spartalizumab, camrelizumab, sintilimab, tislelizumab, toripalimab, dostarlimab, INCMGA00012, AMP-224, AMP-514, KN035, AUNP12, CK-301, CA-170, and BMS-986189.

Suitably, Treg depletor according to the invention and the checkpoint inhibitor may be comprised in a single molecule, such as an antibody that binds to a cell surface marker of a Treg and an immune checkpoint. Thus, in a particular embodiment, the Treg depletor as described herein is a bispecific antibody that binds to a cell surface marker of a Treg and a protein selected from the group consisting of PD-1, PD-L1, B7-1 and B7-2. Suitably, the Treg depletor as described herein may comprise a PD-1 or PD-L1 binding portion of nivolumab, pembrolizumab, atezolizumab, avelumab, durvalumab, cemiplimab, JTX-4014, spartalizumab, camrelizumab, sintilimab, tislelizumab, toripalimab, dostarlimab, INCMGA00012, AMP-224, AMP-514, KNO35, AUNP12, CK-301, CA-170, and BMS-986189.

### BRIEF DESCRIPTION OF THE FIGURES

FIG. 1 illustrates the evaluation by flow cytometry of two VHHs (VHH-01 and VHH-06) derived from llama immunization with mouse CCR8 for their binding to full-length mouse CCR8 versus N-terminal deletion mouse CCR8 overexpressed in Hek293 cells.

FIG. 2 illustrates the evaluation of VHH-Fc-14 for its potential to functionally inhibit the protective activity of ligand CCL1 against dexamethasone-induced apoptosis in BW5147 cells.

FIG. 3 shows the effects on intratumoural Treg depletion by VHH-Fc-43, which is a CCR8 Fc fusion with ADCC activity, as well as isotype control.

FIG. 4 shows the effects on circulating Tregs by VHH-Fc-43 and isotype control.

FIG. 5 shows the in vivo effects of VHH-FC-43 and VHH-16 monotherapies on tumour growth in comparison to isotype and combination therapy with VHH-Fc-43 and VHH-16 in MC38 tumours from day when tumours are inoculated, to the trial endpoint at day 25.

FIG. 6 shows the Kaplan-Meier survival curve for the isotype, VHH-FC-43 and VHH-16 monotherapy, and VHH-Fc-43 and VHH-16 combination therapy treated tumours. Animals were sacrificed when their tumours reached the ethical endpoint of 2000 mm³.

FIG. 7 depicts quantification of the numbers of HEVs found in tumours treated with isotype (day 21), VHH-FC-43 and VHH-16 monotherapy (day 25), and VHH-Fc-43 and VHH-16 combination therapy (day 25) per tumor area. Sections from one tumor each from 3 treated mice for each condition was analyzed, and total tumor area was calculated by outlining the DAPI-positive nuclei using the Zen Blue software program.

FIG. 8 shows “mature” appearing tertiary lymphoid structures (TLSs), identified in tumours treated with VHH-Fc-43 and VHH-16 combined therapy. Arrows show MECA-79 positive HEVs surrounding an organized structure consisting of copious B220 positive B cells.

FIG. 9 shows the in vivo effects of anti-CTLA-4 and VHH-16 monotherapies on tumour growth in comparison to isotype and combination therapy with anti-CTLA-4 and VHH-16 in MC38 tumours from day 0, when tumours are inoculated, to the trial endpoint at day 25. The anti-CTLA-4 used in these experiments is a mAb comprising a mouse IgG2a, thereby enabling the anti-CTLA-4 IgG to deplete Treg cells.

### DETAILED DESCRIPTION OF THE INVENTION

The present invention will be described in the following with respect to particular embodiments and with reference to certain drawings but the invention is not limited thereto.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclature used in connection with, and techniques of, molecular biology, immunology, microbiology, genetics and protein and nucleic acid chemistry described herein are those well-known and commonly used in the art.

As described herein before, the present invention provides a combination comprising a Treg depletor and an LTBR agonist. Such a combination is particularly useful due to the synergistic effect observed when the Treg depletor and the LTBR agonist as defined in the combination of the present invention are administrated as a combined cancer therapy.

### Treg Depletor

As used herein, the term “Treg depletor” denotes a molecule capable of depleting (ablating) a significant portion of a subject's Treg. In some embodiments, the majority of Treg cells are ablated in a subject. In some embodiments, greater than 50%, 60%, 70%, 80%, 90%, 95%, or 99% Treg are ablated in a subject. In a particular embodiment, a Treg depletor binds to a Treg cell and depletes. In a further particular embodiment, a Treg depletor is a molecule capable of binding to a cell surface surface marker of a Treg cell and inducing its depletion through its cytotoxic activity. In a further particular embodiment, the Treg depletor depletes intra-tumoral Tregs to a greater extent than other Tregs, such a tissue-infiltrating Tregs and circulating blood Tregs. In another particular embodiment, the Treg depletor depletes intra-tumoral Tregs to a greater extent than other T cells. In yet another particular embodiment, the Treg depletor depletes intra-tumoral Tregs and increases the ratio of effector T cells over Tregs in the tumor microenvironment, preferably in the tumor. In a particular embodiment, Treg depletion is measured by treating isolated human Tregs or tumor infiltrating lymphocytes with a compound, and if needed in the presence of effector cells like NK cels or PBMC, and analyzing the number of viable Treg cells after treatment, essentially as described in Pablos et al. (BMC Immunology 2005, 6:6 doi:10.1186/1471-2172-6-6). Alternatively, and in one embodiment complementary, Treg depletion may be verified by adding the compound to PBMC and and measure the level of viable Tregs after 4 hrs. Alternatively, Treg depletion is verified through incubation of PBMC with a compound and capturing the cells bound by the compound using magnetic beads, followed by FACS analysis of the non-captured cells essentially as described in Sugiyama et al. (Proc Natl Acad Sci U S A 2013 October 29; 110(44):17945-50. doi: 10.1073/pnas.1316796110). A suitable in vivo assay for determining Treg depletion comprises FACS analysis of tumor infiltrating immune cells after administration of the Treg depleting compound to the mice.

In yet another embodiment, the cell surface marker of a Treg is a marker that is overexpressed on the cell surface of a Treg compared to the expression of the marker on the cell surface of another T cell. In a more particular embodiment, the cell surface marker of a Treg is a marker that is overexpressed on the cell surface of tumour-infiltrating Treg compared to its expression on peripheral Treg cells.

Preferably, the cell surface marker of a Treg is selected from the group consisting of CCR8, CCR4, CTLA4, CD25, TIGIT, OX40, ICOS, CD38, GITR, 4-1BB, NRP1, and LAG-3. In a particular embodiment, the cell surface marker of a Treg is selected from CCR8, CCR4, CD25, TIGIT, and ICOS; preferably CCR8, CD25, and CCR4

Thus, in a particular embodiment, the cell surface marker of a Treg is the CC chemokine receptor 4 (CCR4). CCR4 binding antibodies having cytotoxic activity have been disclosed e.g. in WO2013166500 A1, WO2016057488 A1 and WO2016178779 A1. In a particular embodiment, the Treg depletor for use in the invention is mogamulizumab.

In another particular embodiment, the cell surface marker of a Treg is CTLA4, also known as CTLA-4 or cytotoxic T-lymphocyte-associated protein 4. CTLA4 binding antibodies have been disclosed e.g. in WO2013003761 A1 and WO2017106372 A1. In a particular embodiment, the Treg depletory for use in the invention is ipilimumab or tremelimumab.

In another particular embodiment, the cell surface marker of a Treg is CD25. Interleukin-2 receptor alpha chain (also called CD25) is a protein that in humans is encoded by the IL2RA gene. The interleukin 2 (IL2) receptor alpha (IL2RA) and beta (IL2RB) chains, together with the common gamma chain (IL2RG), constitute the high-affinity IL2 receptor. Suitable CD25 binding antibodies have been disclosed e.g. in WO2017174331 A1, WO2018167104 A1 and WO2019175220 A1, all of which are incorporated herein by reference. In a particular embodiment, the CD25 binding antibody for use in the invention is RG6292, also known as RO7296682.

In another particular embodiment, the cell surface marker of a Treg is TIGIT. TIGIT (also called T cell immunoreceptor with Ig and ITIM domains) is an immune receptor present on some T cells and Natural Killer Cells (NK). It is also identified as WUCAM and Vstm3. Suitable TIGIT binding antibodies for use in the invention have been disclosed e.g. in WO2015009856 A2, WO2016028656 A1, WO2016106302 A1, WO2017053748 A2, WO2017152088 A1, and WO2019023504 A1. In a further embodiment, the Treg depletor is tiragolumab; an antibody comprising a heavy chain variable domain comprising the amino acid sequence of SEQ ID NO: 221 and a light chain variable domain comprising the amino acid sequence of SEQ ID NO: 222 of WO2019023504 A1; or an antibody comprising a heavy chain variable domain comprising the amino acid sequence of SEQ ID NO: 219 and a light chain variable domain comprising the amino acid sequence of SEQ ID NO: 220 of WO2019023504 A1.

In another particular embodiment, the cell surface marker of a Treg is OX40. OX40 (also known as Tumor necrosis factor receptor superfamily, member 4 (TNFRSF4) or CD134) is a secondary co-stimulatory molecule. Suitable OX40 binding antibodies for use in the invention have been disclosed e.g. in WO2018031400 A1, WO2007062245 A2, WO2018202649 A1, WO2016179517 A1 and WO2018112346 A1. In a particular embodiment, the Treg depletor is selected from KHK4083, ATOR-1015, INCAGN01949, and ABBV-368. In another particular embodiment, the Treg depletor is an antibody selected from:
an antibody comprising a heavy chain variable region comprising an amino acid sequence from the amino acid at position 20 to 141 of SEQ ID NO:9 of WO2007062245 A2, and a light chain variable region comprising an amino acid sequence from the amino acid at position 21 to 129 of SEQ ID NO:10 of WO2007062245 A2;
an antibody comprising a heavy chain variable region comprising an amino acid sequence of SEQ ID NO:91 of WO2018202649 A1, and a light chain variable region comprising an amino acid sequence of SEQ IDNO:89 of WO2018202649 A1;
an antibody comprising a heavy chain variable region comprising an amino acid sequence of SEQ ID NO:16 of WO2016179517 A1, and a light chain variable region comprising an amino acid sequence of SEQ IDNO:15 of WO2016179517 A1; and
antibody Hu3738 of WO2018112346 A1.

In another particular embodiment, the cell surface marker of a Treg is ICOS. ICOS (also known as Inducible T-cell COStimulator or CD278) is an immune checkpoint protein encoded by the ICOS gene. It is a CD28-superfamily costimulatory molecules that is expressed on activated T cells. Suitable ICOS binding antibodies for use in the invention have been disclosed e.g. in WO2008137915 A2, WO2016154177 A2, WO2012131004 A2, WO2018029474 A2, and WO2018187613 A2. In a particular embodiment, the Treg depletor is selected from KY-11044, KY-1055, XmAb23104, vopratelimab, and MEDI-570. In another particular embodiment, the Treg depletor is an antibody selected from:
vopratelimab;
an antibody comprising a heavy chain variable region comprising an amino acid sequence of SEQ ID NO:408 of WO2018029474 A2, and a light chain variable region comprising an amino acid sequence of SEQ ID NO:415 of WO2018029474 A2; and
an antibody comprising a heavy chain variable region comprising an amino acid sequence of SEQ ID NO:7 of WO2008137915A2, and a light chain variable region comprising an amino acid sequence of SEQ ID NO:2 of WO2008137915A2.

In yet another particular embodiment, the cell surface marker of a Treg is CD38. CD38 (Cluster of Differentiation 38, also known as cyclic ADP ribose hydrolase) is a glycoprotein found on the surface of many immune cells, including CD4+, CD8+, B lymphocytes and natural killer cells. CD38 also functions in cell adhesion, signal transduction and calcium signaling. Suitable CD38 binding antibodies for use in the invention have e.g. been disclosed in WO2016210223 A1, WO2012092616 A1, WO2008047242 A2, and WO2015066450 A1. In another particular embodiment, the Treg depletor is an antibody selected from:
daratumumab;
isatuximab; and
an antibody comprising a heavy chain variable region comprising an amino acid sequence of SEQ ID NO:9 of WO2012092616 A1, and a light chain variable region comprising an amino acid sequence of SEQ ID NO:10 of WO2012092616 A1.

In another particular embodiment, the cell surface marker of a Treg is GITR. GITR (glucocorticoid-induced TNFR-related protein, also known as Tumor necrosis factor receptor superfamily member 18 (TNFRSF18) or as activation-inducible TNFR family receptor (AITR)) is also a co-stimulatory immune checkpoint molecule that plays a key role in dominant immunological self-tolerance maintained by CD25+/CD4+ regulatory T cells. Suitable GITR binding antibodies for use in the invention have been disclosed e.g. in WO2015187835 A2, WO2016054638 A1, WO2016081746 A2, WO2015184099 A1, and WO2016057846 A1. In another particular embodiment, the Treg depletor is an antibody selected from:
an antibody having the heavy chain and light chain variable regions of the antibody 28F3.IgG1 of WO2015187835 A2;
an antibody comprising a heavy chain variable region comprising an amino acid sequence of SEQ ID NO:206 of WO2015184099 A1, and a light chain variable region comprising an amino acid sequence of SEQ ID NO:208 of WO2015184099 A1;
an antibody comprising a heavy chain variable region comprising an amino acid sequence of SEQ ID NO:99 of WO2016057846 A1, and a light chain variable region comprising an amino acid sequence of SEQ ID NO:7 of WO2016057846 99 A1.

In another particular embodiment, the cell surface marker of a Treg is 4-1BB. 4-1BB (also known as tumor necrosis factor receptor superfamily member 9 (TNFRSF9), CD137 and induced by lymphocyte activation (ILA)) is also a co-stimulatory immune checkpoint molecule. Suitable molecules include urelumab and utomilumab and derivatives thereof with increased cytotoxic activity, especially ADCC activity.

In another particular embodiment, the cell surface marker of a Treg is NRP1. NRP1 (also known as neuropilin-1) is a membrane-bound coreceptor to a tyrosine kinase receptor for both VEGF and semaphorin family members. NRP1 plays versatile roles in angiogenesis, axon guidance, cell survival, migration and invasion and is highly expressed on Tregs. Suitable molecules for use in the invention include the antibodies those disclosed in WO2007056470, WO2012006503 A1, WO2014058915 A2, and WO2018119171 A1, as well as derivatives thereof with increased cytotoxic activity, especially ADCC activity. In a particular embodiment, the Treg depletor is vesencumab. In another particular embodiment, the Treg depletor comprises the heavy chain and light chain variable regions of MAB12 of WO2018119171 A1.

In yet another particular embodiment, the cell surface marker of a Treg is LAG3. LAG3 (Lymphocyte-activation gene 3, also known as CD223) is an immune checkpoint receptor. Suitable LAG3 binding antibodies for use in the invention have been disclosed e.g. in WO2014140180 A1; WO2014008218 A1; US20160176965 A1; WO2016028672 A1; and WO2010019570 A2. In another particular embodiment, the Treg depletor is an antibody comprising a heavy chain variable region comprising an amino acid sequence of SEQ ID NO:9 of WO2014140180 A1, and a light chain variable region comprising an amino acid sequence of SEQ ID NO:4 of WO2014140180 A1.

More preferably, the cell surface marker of a Treg is CCR8. CCR8 is a member of the beta-chemokine receptor family which is predicted to be a seven transmembrane protein similar to G-coupled receptors. Identified ligands of CCR8 include its natural cognate ligand CCL1 (I-309). The inventors have found that Treg modulation through targeting CCR8 allows to specifically deplete tumour-infiltrating Treg cells while preserving tumour-reactive effector T cells and peripheral Treg cells (e.g. circulating blood Treg cells).

“Specific binding”, “bind specifically”, and “specifically bind” is particularly understood to mean that the Treg depletor has a dissociation constant (K_{d}) for the marker/antigen of interest of less than about 10⁻⁶M, 10⁻⁷M, 10⁻⁸M, 10⁻⁹M, 10⁻¹⁰M, 10⁻¹¹M, 10¹²M or 10⁻¹³M. In a preferred embodiment, the dissociation constant is less than 10⁻⁸M, for instance in the range of 10⁻⁹M, 10⁻¹⁰M, 10⁻¹¹M, 10⁻¹²M or 10⁻¹³M. Treg depletor affinities towards membrane targets may be determined by a surface plasmon resonance based assay (such as the BIAcore assay as described in PCT Application Publication No. WO2005/012359) using viral like particles; cellular enzyme-linked immunoabsorbent assay (ELISA); and fluorescent activated cell sorting (FACS) read outs for example. A preferred method for determining apparent Kd or EC50 values is by using FACS at 21° C. with cells overexpressing the marker, in particular overexpressing huCCR8.

As will be understood by the skilled person, in principle any type of Treg depletor that binds to a cell surface marker of a Treg can be used in the present invention and different types of Treg depletors are readily available to the skilled person or can be generated using the typical knowledge in the art. In a particular embodiment, the binding moiety of the Treg depletor is proteinaceous, more particularly a Treg depleting polypeptide. In a further embodiment, the binding moiety of the Treg depletor is antibody based or non-antibody based, preferably antibody based. Non-antibody based Treg depletors include, but are not limited to, affibodies, Kunitz domain peptides, monobodies (adnectins), anticalins, designed ankyrin repeat domains (DARPins), centyrins, fynomers, avimers; affilins; affitins, peptides and the like.

As described herein, the terms “antibody”, “antibody fragment” and “active antibody fragment” refer to a protein comprising an immunoglobulin (Ig) domain or an antigen-binding domain capable of specifically binding the antigen, in particular the CCR8 protein. “Antibodies” can further be intact immunoglobulins derived from natural sources or from recombinant sources and can be immunoreactive portions of intact immunoglobulins. Antibodies may be multimers, such as tetramers, of immunoglobulin molecules. In a preferred embodiment, the Treg depletor comprises a Treg depleting moiety, in particular a CCR8 binding moiety, being an antibody or active antibody fragment. In a further aspect of the invention, the Treg depletor is an antibody. In a further aspect of the invention the antibody is monoclonal. The antibody may additionally or alternatively be humanised or human. In a further aspect, the antibody is human, or in any case an antibody that has a format and features allowing its use and administration in human subjects. Antibodies may be derived from any species, including but not limited to mouse, rat, chicken, rabbit, goat, bovine, non-human primate, human, dromedary, camel, llama, alpaca, and shark.

The term “antigen-binding fragment” is intended to refer to an antigen-binding portion of said intact polyclonal or monoclonal antibodies that retains the ability to specifically bind to a target antigen or a single chain thereof, fusion proteins comprising an antibody, and any other modified configuration of the immunoglobulin molecule that comprises an antigen recognition site. The antigen-binding fragment comprises, but not limited to Fab; Fab′; F(ab′)₂; a Fc fragment; a single domain antibody (sdAb or dAb) fragment. These fragments are derived from intact antibodies by using conventional methods in the art, for example by proteolytic cleavage with enzymes such as papain (to produce Fab fragments) or pepsin (to produce F(ab′)₂ fragments). As used herein, antigen-binding fragment also refers to fusion proteins comprising heavy and/or light chain variable regions, such as single-chain variable fragments (scFv).

As used herein, the term “monoclonal antibody” refers to an antibody composition having a homogeneous antibody population. It is understood that monoclonal antibodies are highly specific, being directed against a single antigenic site. Furthermore, in contrast to conventional antibody (polyclonal) preparations which typically include different antibodies directed against different determinants (epitopes), each monoclonal antibody is directed against a single determinant on the antigen. The Treg depletors of the invention preferably comprise a monoclonal antibody moiety that binds to a cell surface marker of a Treg, in particular to CCR8 or CTLA4, more in particular to CCR8.

As used herein, the term “humanized antibody” refers to an antibody produced by molecular modeling techniques to identify an optimal combination of human and non-human (such as mouse or rabbits) antibody sequences, that is, a combination in which the human content of the antibody is maximized while causing little or no loss of the binding affinity attributable to the variable region of the non-human antibody. For example, a humanized antibody, also known as a chimeric antibody comprises the amino acid sequence of a human framework region and of a constant region from a human antibody to “humanize” or render non-immunogenic the complementarity determining regions (CDRs) from a non-human antibody.

As used herein, the term “human antibody” means an antibody having an amino acid sequence corresponding to that of an antibody that can be produced by a human and/or which has been made using any of the techniques for making human antibodies known to a skilled person in the art or disclosed herein. It is also understood that the term “human antibody” encompasses antibodies comprising at least one human heavy chain polypeptide or at least one human light chain polypeptide. One such example is an antibody comprising murine light chain and human heavy chain polypeptides.

In one aspect of the invention, the Treg depletor comprises an active antibody fragment. The term “active antibody fragment” refers to a portion of any antibody or antibody-like structure that by itself has high affinity for an antigenic determinant, or epitope, and contains one or more antigen-binding sites, e.g. complementary-determining-regions (CDRs), accounting for such specificity. Non-limiting examples include immunoglobulin domains, Fab, F(ab)′2, scFv, heavy-light chain dimers, immunoglobulin single variable domains, single domain antibodies (sdAb or dAb), Nanobodies®, and single chain structures, such as complete light chain or complete heavy chain, as well as antibody constant domains that have been engineered to bind to an antigen. An additional requirement for the “activity” of said fragments in the light of the present invention is that said fragments are capable of binding a cell surface marker of a Treg, in particular CCR8. The term “immunoglobulin (Ig) domain” or more specifically “immunoglobulin variable domain” (abbreviated as “IVD”) means an immunoglobulin domain essentially consisting of framework regions interrupted by complementary determining regions. Typically, immunoglobulin domains consist essentially of four “framework regions” which are referred in the art and below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred in the art and herein below as “complementarity determining region 1” or “CDR1”; as “complementarity determining region 2” or “CDR2”; and as “complementarity determining region 3” or “CDR3”, respectively. Thus the general structure or sequence of an immunoglobulin variable domain can be indicated as follows: FR1-CDR1-FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, an heavy chain variable domain (VH) and a light chain variable domain (VL) interact to form an antigen binding site. In this case the complementary determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as IgG, IgM, IgA, IgD or IgE molecule; known in the art) or of a Fab fragment, a F(ab′)2 fragment, an Fv fragment such as a disulphide linked Fv or scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. A single domain antibody (sdAb) as used herein, refers to a protein with an amino acid sequence comprising 4 framework regions (FR) and 3 complementarity determining regions (CDRs) according to the format FR1-CDR1-FR2-CDR2-FR3-CDR3-FR4. Single domain antibodies of this invention are equivalent to “immunoglobulin single variable domains” (abbreviated as “ISVD”) and refers to molecules wherein the antigen binding site is present on, and formed by, a single immunoglobulin domain. This sets single domain antibodies apart from “conventional” antibodies or their fragments, wherein two immunoglobulin domains, in particular two variable domains interact to form an antigen binding site. The binding site of a single domain antibody is formed by a single VH/VHH or VL domain. Hence, the antigen binding site of a single domain antibody is formed by no more than 3 CDRs. As such a single domain may be a light chain variable domain sequence. (e.g. a VL-sequence) or a suitable fragment thereof; or a heavy chain variable domain sequence (e.g. a VH-sequence or VHH sequence) or a suitable fragment thereof; as long as it is capable of forming a single antigen binding unit (i.e., a functional antigen binding unit that essentially consists of a single variable domain, such that the single antigen binding domain does not need to interact with another variable domain to form a functional antigen binding unit).

Thus, in one embodiment, the Treg depletor binding to a cell surface marker of a Treg and having cytotoxic activity as detailed above, comprises at least one single domain antibody moiety. Preferably, the Treg depletor binding to a cell surface marker of a Treg and having cytotoxic activity comprises at least two single domain antibody moieties.

In a further embodiment of the present invention, the Treg depletor, as detailed above, comprises at least one Fc region moiety and at least two single domain antibody moieties that bind to a cell surface marker of a Treg, in particular to CCR8. Preferably, the Treg depletor is a genetically engineered polypeptide that comprises at least one Fc region moiety and at least two single domain antibody moieties that bind to a cell surface marker of a Treg, in particular to CCR8, joined together by a peptide linker. The amino acid sequence of the Fc region moiety and/or the single domain antibody moiety region(s) may be humanized to reduce immunogenicity for humans.

In particular, the single domain antibody may be a Nanobody® (as defined herein) or a suitable fragment thereof (Note: Nanobody®, Nanobodies® and Nanoclone® are registered trademarks of Ablynx N.V., a Sanofi Company). For general description of Nanobodies ° reference is made to the further description below, and described in the prior art such as e.g. WO2008/020079. “VHH domains”, also known as VHHs, VHH antibody fragments and VHH antibodies, have originally been described as the antigen binding immunoglobulin (Ig) (variable) domain of “heavy chain antibodies” (i.e. of “antibodies devoid of light chains”; see e.g. Hamers-Casterman et al., Nature 363:446-8 (1993)). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobodies®, reference is made to the review article by Muyldermans (Reviews in Molecular Biotechnology 74: 277-302, 2001), as well as to the following patent applications, which are mentioned as general background art: WO 94/04678, WO and WO 96/34103 of the Vrije Universiteit Brussel; WO 94/25591, WO 99/37681, WO 00/40968,WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody® (in particular VHH sequences and partially humanized Nanobody) can in particular be characterized by the presence of one or more “Hallmark residues” in one or more of the framework sequences. A further description of the Nanobody®, including humanization and/or camelization of Nanobody, as well as other modifications, parts or fragments, derivatives or “Nanobody fusions”, multivalent or multispecific constructs (including some non-limiting examples of linker sequences) and different modifications to increase the half-life of the Nanobody® and their preparations can be found e.g. in WO 08/101985 and WO 08/142164. VHHs and Nanobodies® are among the smallest antigen binding fragment that completely retains the binding affinity and specificity of a full-length antibody (see e.g. Greenberg et al., Nature 374:168-73 (1995); Hassanzadeh-Ghassabeh et al., Nanomedicine (Lond), 8:1013-26 (2013)).

Furthermore, as for full-size antibodies, single variable domains such as VHHs and Nanobodies® can be subjected to humanization, i.e. increase the degree of sequence identity with the closest human germline sequence. In particular, humanized immunoglobulin single variable domains, such as VHHs and Nanobodies® may be single domain antibodies in which at least one single amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person.

Humanized single domain antibodies, in particular VHHs and Nanobodies®, may have several advantages, such as a reduced immunogenicity, compared to the corresponding naturally occurring VHH domains. By humanized is meant mutated so that immunogenicity upon administration in human patients is minor or non-existent. The humanizing substitutions should be chosen such that the resulting humanized amino acid sequence and/or VHH still retains the favourable properties of the VHH, such as the antigen-binding capacity. Based on the description provided herein, the skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also a subset of human germline alleles most homologous to the target sequence may be aligned as starting point to identify suitable humanisation residues. Alternatively, the VHH is analyzed to identify its closest homologue in the human alleles, and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization.

Suitable mutations, in particular substitutions, can be introduced during humanization to generate a polypeptide with reduced binding to pre-existing antibodies (reference is made for example to WO 2012/175741 and WO2015/173325), for example at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined below) or at one or more other framework residues (i.e. non-Hallmark residues) or any suitable combination thereof. Depending on the host organism used to express the amino acid sequence, VHH or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation.

In some cases, at least one of the typical Camelidae hallmark residues with hydrophilic characteristics at position 37, 44, 45 and/or 47 is replaced (see WO2008/020079 Table A-03). Another example of humanization includes substitution of residues in FR 1, such as position 1, 5, 11, 14, 16, and/or 28; in FR3, such as positions 73, 74, 75, 76, 78, 79, 82b, 83, 84, 93 and/or 94; and in FR4, such as position 103, 104, 108 and/or 111 (see WO2008/020079 Tables A-05-A08; all numbering according to the Kabat).

In one aspect of the invention, the Treg depletor as defined in the combination of the present invention is monospecific. As discussed further below, in an alternative aspect the Treg depletor of the invention is bispecific.

As used herein, “bispecific” refers to a Treg depletor having the capacity to bind two distinct epitopes either on a single antigen or polypeptide, or on two different antigens or polypeptides.

Bispecific Treg depletors of the present invention as discussed herein can be produced via biological methods, such as somatic hybridization; or genetic methods, such as the expression of a non-native DNA sequence encoding the desired structure in a cell line or in an organism; chemical methods (e.g. by chemical coupling, genetic fusion, noncovalent associated or otherwise to one or more molecular entities, such as another binder of fragment thereof); or combination thereof.

The technologies and products that allow producing monospecific or bispecific Treg depletors are known in the art, as extensively reviewed in the literature, also with respect to alternative formats, Treg depletor-drug conjugates, Treg depletor design methods, in vitro screening methods, constant regions, post-translational and chemical modifications, improved feature for triggering cancer cell death such as Fc domain engineering (Tiller K and Tessier P, Annu Rev Biomed Eng. 17:191-216 (2015); Speiss C et al.,

Molecular Immunology 67:95-106 (2015); Weiner G, Nat Rev Cancer, 15:361-370 (2015); Fan G et al., J Hematol Oncol 8:130 (2015)).

As used herein, “epitope” or “antigenic determinant” refers to a site on an antigen to which a Treg depletor, such as an antibody, binds. As is well known in the art, epitopes can be formed both from contiguous amino acids (linear epitope) or non-contiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes are well known in the art and include, for example, x-ray crystallography and 2-D nuclear magnetic resonance. See, for example, Epitope Mapping Protocols in Methods in Molecular Biology, Vol. 66, Glenn E. Morris, Ed (1996).

Further according to the invention, the Treg depletor as defined in the combination of the present invention binds to a cell surface marker of a Treg cell and has cytotoxic activity. “Cytotoxicity” or “cytotoxic activity” as used herein refers to the ability of a Treg depletor to be toxic to a cell that it is bound to. As is clear to the skilled person from the description of the invention, any type of cytotoxicity can be used in the context of the invention. Of importance is the ability of the Treg depletor of the invention to bind a cell surface marker of a Treg cell, such as CCR8, and to cause toxicity to the cell that it is bound to. Cytotoxicity can be direct cytotoxicity, wherein the Treg depletor itself directly damages the cell (e.g. because it comprises a chemotherapeutic payload) or it can be indirect, wherein the Treg depletor induces extracellular mechanisms that cause damage to the cell (e.g. an antibody that induces antibody-dependent cellular activity). More in particular, the Treg depletor of the invention can signal the immune system to destroy or eliminate the cell it is bound to or the Treg depletor can carry a cytotoxic payload to destroy the cell it is bound to. In particular, the cytotoxic activity is caused by the presence of cytotoxic moiety. Examples of such cytotoxic moieties includes moieties which induce antibody-dependent cellular activity (ADCC), induce complement-dependent cytotoxicity (CDC), induce antibody-dependent cellular phagocytosis (ADCP), bind to and activate T-cells, or comprise a cytotoxic payload. Most preferably, said cytotoxic moiety induces antibody-dependent cellular activity (ADCC).

Antibody-dependent cellular cytotoxicity (ADCC) refers to a cell-mediated reaction in which non-specific cytotoxic cells that express Fc receptors recognize Treg depletors on a target cell and subsequently cause lysis of the target cell. Examples of non-specific cytotoxic cells that express Fc receptors include natural killer cells, neutrophils and macrophages.

Complement-dependent cytotoxicity (CDC) refers to the lysis of a target in the presence of complement. The complement activation pathway is initiated by the binding of the first component of the complement system (C1q) to a Treg depletor complexed with a cognate antigen.

Antibody-dependent cellular phagocytosis (ADCP) refers to a cell-mediated reaction in which phagocytes (such as macrophages) that express Fc receptors recognize Treg depletors on a target cell and thereby lead to phagocytosis of the target cell.

CDC, ADCC and ADCP can be measured using assays that are known in the art (Vafa et al. Methods 2014 Jan. 1; 65(1):114-26 (2013)).

The cytotoxic activity may also be caused by a cytotoxic moiety that binds to and activates cytotoxic T-cells or T helper cells, for example because the cytotoxic moiety binds to a cytotoxic T-cell or T helper cell marker that is distinct from the cell surface marker of a Treg, preferably that is distinct from CCR8, and the binding results in activation of said cytotoxic T-cell or T helper cell. Activation of the cytotoxic T-cell or T helper cell induces the cytotoxic activity of the cytotoxic T-cell or T helper cell against the cell on which the Treg depletor of the invention is bound. Therefore, in a particular embodiment, the

Treg depletor of the invention binds to a cell surface marker of a Treg, preferably to CCR8, and binds to and activates cytotoxic T-cell or T helper cell. For example, the cytotoxic moiety may bind to CD3. In a further embodiment, the cytotoxic moiety comprises an antibody or antigen-binding fragment thereof that binds to CD3. Thus, the Treg depletor of the invention may bind to a cell surface marker of Treg, preferably to CCR8, and CD3. Such a Treg depletor binds to intratumoural Tregs and directs the cytotoxic activity of T-cells to these Tregs, thereby depleting them from the tumour environment. In a particular embodiment, the Treg depeletor of the invention comprises a moiety that binds to a cell surface marker of a Treg, in particular to CCR8, and a moiety that binds to CD3, wherein at least one moiety is antibody based, particularly wherein both moieties are antibody based. Therefore, in a particular embodiment, the present invention provides a bispecific construct comprising an antibody or antigen-binding fragment thereof that specifically binds to a cell surface marker of a Treg, preferably to CCR8, and an antibody or antigen-binding fragment thereof that specifically binds to CD3.

A cytotoxic payload refers to any molecular entity that causes a direct damaging effect on the cell that is contacted with the cytotoxic payload. Cytotoxic payloads are known to the persons skilled in the art. In a particular embodiment, the cytotoxic payload is a chemical entity. Particular examples of such cytotoxic payloads include toxins, chemotherapeutic agents and radioisotopes or radionuclides. In a further embodiment, the cytotoxic payload comprises an agent selected from the group consisting of alkylating agents, anthracyclines, cytoskeletal disruptors, epothilones, histone deacetylase inhibitors, inhibitors of topoisomerase I, inhibitors of topoisomerase II, kinase inhibitors, nucleotide analogues and precursor analogues, peptide antibiotics, platinum-based agents, retinoids, vinca alkaloids and derivatives, peptide or small molecule toxins, and radioisotopes. Chemical entities can be coupled to proteinaceous inhibitors, e.g. antibodies or antigen-binding fragments, using techniques known in the art. Such coupling can be covalent or non-covalent and the coupling can be labile or reversible.

As is well known in the field, the Fc region of IgG antibodies interacts with several cellular Fcγ receptors (FcγR) to stimulate and regulate downstream effector mechanisms. There are five activating receptors, namely FcγRI (CD64), FcγRIIa (CD32a), FcγRIIc (CD32c), FcγRIIIa (CD16a) and FcγRIIIb (CD16b), and one inhibitory receptor FcγRIIb (CD32b). The communication of IgG antibodies with the immune system is controlled and mediated by FcγRs, which relay the information sensed and gathered by antibodies to the immune system, providing a link between the innate and adaptive immune systems, and particularly in the context of biotherapeutics (Hayes J et al., 2016. J Inflamm Res 9: 209-219).

IgG subclasses vary in their ability to bind to FcγR and this differential binding determines their ability to elicit a range of functional responses. For example, in humans, FcγRIIIa is the major receptor involved in the activation of antibody-dependent cell-mediated cytotoxicity (ADCC) and IgG3 followed closely by IgG1 display the highest affinities for this receptor, reflecting their ability to potently induce ADCC. Whilst IgG2 have been shown to have weaker binding for this receptor, Treg depletors having the human IgG2 isotype have also been found to efficiently deplete Tregs.

In a preferred embodiment of the invention, the Treg depletor of the invention induces antibody effector function, in particular antibody effector function in human. In a particular embodiment, the Treg depletor of the invention binds FcγR with high affinity, preferably an activating receptor with high affinity. Preferably the Treg depletor binds FcγRI and/or FcγRIIa and/or FcγRIIIa with high affinity. Particularly preferably, the Treg depletor binds to FcγRIIIa. In a particular embodiment, the Treg depletor binds to at least one activating Fcγ receptor with a dissociation constant of less than about 10⁻⁶M, 10⁻⁷M, 10⁻⁸M, 10⁻⁹M, 10⁻¹⁰M, 10⁻¹¹M, 10⁻¹²M or 10⁻¹³M. FcγR binding can be obtained through several means. For example, the cytotoxic moiety may comprise a fragment crystallisable (Fc) region moiety or it may comprise a binding part, such as an antibody or antigen-binding part thereof that specifically binds to an FcγR.

Therefore, in one embodiment, the cytotoxic moiety comprises a fragment crystallisable (Fc) region moiety. Within the context of the present invention the term “fragment crystallisable (Fc) region moiety” refers to the crystallisable fragment of an immunoglobulin molecule composed of the constant regions of the heavy chains and responsible for the binding to antibody Fc receptors and some other proteins of the complement system, thereby inducing ADCC, CDC, and/or ADCP activity.

In one embodiment, the Fc region moiety has been engineered to increase ADCC, CDC and/or ADCP activity.

ADCC may be increased by methods that reduce or eliminate the fucose moiety from the Fc moiety glycan and/or through introduction of specific mutations on the Fc region of an immunoglobulin, such as IgG1 (e.g. S298A/E333/K334A, S239D/I332E/A330L or G236A/S239D/A330L/I332E) (Lazar et al. Proc Natl Acad Sci USA 103:2005-2010 (2006); Smith et al. Proc Natl Acad Sci USA 209:6181-6 (2012)). ADCP may also be increased by the introduction of specific mutations on the Fc portion of human IgG (Richards et al. Mol Cancer Ther 7:2517-27 (2008)). Methods for engineering binders for increased ADCC, CDC and ADCP activity have been described in Saunders (Frontiers in Immunology 2019, 1296) and Wang et al. (Protein Cell 2019, 9:63-73).

In a particular embodiment of the present invention, the Treg depletor comprising an Fc region moiety is optimized to elicit an ADCC response, that is to say the ADCC response is enhanced, increased or improved relative to other ones comprising an Fc region moiety, including those that do not inhibit the binding of a ligand, in particular of CCL1, to its receptor, in particular to CCR8, and for example, unmodified anti-CCR8 monoclonal antibodies. In a preferred embodiment, the Treg depletor has been engineered to elicit an enhanced ADCC response.

In a preferred embodiment of the present invention, the Treg depletor comprising an Fc region moiety is optimized to elicit an ADCP response, that is to say the ADCP response is enhanced, increased or improved relative to other ones comprising an Fc region moiety, including those that do not inhibit the binding of a ligand, in particular of CCL1, to its receptor, in particular to CCR8 and, for example, unmodified anti-CCR8 monoclonal antibodies.

In another embodiment, the cytotoxic moiety comprises a moiety that binds to an Fc gamma receptor. More in particular binds to and activates an FcγR, in particular an activating receptor, such as FcγRI and/or FcγRIIa and/or FcγRIIIa, especially FcγRIIIa. The moiety that binds to an FcγR may be antibody based or non-antibody based as described herein before. If antibody based, the moiety may bind the FcγR through its variable region.

In a particular embodiment, the Treg depletor of the present invention is a CCR8 binder. As described herein, the term “binder” of a specific antigen denotes a molecule capable of specific binding to said antigen. The CCR8 binder as used herein refers to a molecule capable of specifically binding to CCR8. Such a binder is also referred to herein as a “CCR8 binder”.

Thus, in a particular embodiment, the CCR8 binder is a monoclonal antibody having ADCC activity. Such antibodies are known in the art, for example from WO2020138489 A1, which is included herein by reference. In a particular embodiment, the CCR8 binder for the present invention is selected from an antibody disclosed in WO2020138489 A1, in particular an antibody as presented in the claims of WO2020138489 A1. In a further embodiment, the CCR8 binder for the present invention is selected from a humanized antibody disclosed in WO2020138489 A1, in particular a humanized antibody as presented in the claims of WO2020138489 A1. In another particular embodiment, the CCR8 binder for the present invention is antibody 10A11, 2C7 or 19D7 from WO2020138489 A1 or its humanized variant; in particular 10A11 or its humanized variant; more in particular the humanized 10A11 antibody. In another particular embodiment, it is 19D7 and more preferably the humanized 19D7 antibody.

In one preferred embodiment, the CCR8 binder for the present invention is an anti-CCR8 antibody comprising a light chain variable region comprising SEQ ID NO: 59 and heavy chain variable region comprising SEQ ID NO: 41 of WO2020138489 A1. In a further embodiment, the light chain constant region comprises SEQ ID NO: 52 and the heavy chain constant region comprises SEQ ID NO: 53 of WO2020138489 A1.

In a particular embodiment, the CCR8 binder is an anti-CCR8 antibody, which is in particular an IgG antibody, more in particular, an IgG1 or IgG4.

In a particular aspect of the invention, the Treg depletor is a non-blocking binder. Benefits may include reduced side effects on the intestinal and/or skin Treg populations, and the absence of or a lowered inhibition of dendritic cell migration towards lymph nodes. It has furthermore been observed that Treg depletion using blocking Treg depletors, such as non-blocking CCR8 binders, especially in combination with checkpoint inhibition such as PD-1/PD-L1 inhibitors, increases neutrophils in the tumour microenvironment. In this aspect of the invention, the non-blocking Treg depletor, such as a non-blocking CCR8 binder, may have a lesser effect on neutrophil increase, thereby providing a greater anti-tumour efficacy.

A “non-blocking” binder means that it does not block or substantially block the binding of a ligand to the cell surface marker. For example, a non-blocking CCR8 binder does not block binding of a CCR8 ligand, to the CCR8 protein. In a further embodiment, the Treg depletor is a binder that does not modulate the activation of the cell surface marker that it binds to. In such embodiment, the Treg depletor is not an agonising or antagonising binder. Therefore, in such embodiment, the Treg depletor is not an agonising or antagonising antibody.

Preferably, the non-blocking CCR8 binder does not block the binding of at least one ligand selected from CCL1, CCL8, CCL16, and CCL18 to CCR8, in particular it does not block binding of CCL1 or CCL18 to CCR8, preferably it does not block the binding of CCL1 to CCR8.

Blockade of ligand binding to a marker, in particular to CCR8, may be determined by methods known in the art. Examples thereof include, but are not limited to, the measurement of the binding of a ligand such as CCL1 to CCR8, the migration of CCR8-expressing cells towards a ligand such as CCL1, increase in intracellular Ca²⁺ levels by a CCR8 ligand such as CCL1, rescue from dexamethasone-induced apoptosis by a ligand such as CCL1, and variation in the expression of a gene sensitive to CCR8 ligand stimulation, such as CCL1 stimulation.

References to “non-blocking”, “non-ligand blocking”, “does not block” or “without blocking” and the like include embodiments wherein the non-blocking Treg depletor of the invention does not block or does not substantially block the signalling of a ligand via the Treg cell surface marker marker. That is, the non-blocking Treg depletor inhibits less than 50% of ligand signalling compared to ligand signalling in the absence of the Treg depletor. In particular embodiments of the invention as described herein, the non-blocking Treg depletor inhibits less than 40%, 35%, 30%, preferably less than about 25% of ligand signalling compared to ligand signalling in the absence of the Treg depletor. In a particular embodiment, the percentage of ligand signalling is measured at a Treg depletor molar concentration that is at least 10, in particular at least 50, more in particular at least 100 times the binding EC50 of the Treg depletor to the cell surface marker. In another embodiment, the percentage of ligand signalling is measured at a Treg depletor, e.g. a CCR8 binder, molar concentration that is at least 10, in particular at least 50, more in particular at least 100 times the molar concentration of the ligand.

Non-blocking Treg depletors, in particular non-blocking CCR8 binders, allow binding of the cell surface marker, in particular of CCR8, without interfering with the binding of at least one ligand to the cell surface marker, in particular to CCR8, or without substantially interfering with the binding of at least one ligand to the marker, in particular to CCR8. Ligand signalling, e.g. CCL1 signalling, via the marker, e.g. CCR8, may be measured by methods as discussed in the Examples and as known in the art. Comparison of ligand signalling in the presence and absence of the Treg depletor, in particular of the CCR8 binder, can occur under the same or substantially the same conditions.

In some embodiments, CCR8 signalling can be determined by measuring the cAMP release.

Specifically, CHO-K1 cells stably expressing recombinant (human) CCR8 receptor (such as FAST-065C available from EuroscreenFAST) are suspended in an assay buffer of KRH: 5 mM KCl, 1.25 mM MgSO4, 124 mM NaCl, 25 mM HEPES, 13.3 mM Glucose, 1.25 mM KH2PO4, 1.45 mM CaCl2, 0.5 g/l BSA, supplemented with 1mM IBMX. The CCR8 binder is added at a concentration of 100 nM and incubated for 30 minutes at 21° C. A mixture of 5 μM forskolin and (human) CCL1 in assay buffer is added to reach a final assay concentration of 5 nM CCL1. The assay mixture is then incubated for 30 minutes at 21° C. After addition of a lysis buffer and 1 hour incubation, the concentration of cAMP is measured. cAMP can be measured by e.g. determining fluorescence levels, such as with the HTRF kit from Cisbio using manufacturer assay conditions (catalogue #62AM9PE). A non-blocking Treg depletor leads to a change of less than 50% of the amount of cAMP compared to a control that lacks the binder. In particular less than 40%, more in particular less than 30%, such as less than 20%. Preferably, a non-blocking Treg depletor leads to a change of less than 10%, more preferably less than 5% of cAMP compared to control.

Techniques for generating non-blocking Treg depletors, including but not limited to non-blocking CCR8 binders, are available to the person skilled in the art. As non-limiting example, antibodies can be generated through immunization using cell surface marker antigens comprising full length surface marker marker or surface marker marker fragments and generated antibodies can be screened for the absence of the surface marker marker blocking activity. In a particular embodiment, antibodies are generated through immunization using surface marker marker fragments that are not involved in ligand binding. Non-blocking antibodies may be obtained through immunization with marker fragments, in particular CCR8 fragments, derived from the N-terminal region, in particular the N-terminal extracellular region which is not located between transmembrane domains. Therefore, in a particular embodiment, the Treg depletor of the invention binds CCR8 at the N-terminal region of the marker. In one particular embodiment, the Treg depletor binds to the N-terminal region of a CCR8 and one or more extracellular loops located between the transmembrane domains of CCR8. In another embodiment, the Treg depletor binds to the N-terminal region of CCR8, and doesn't bind to extracellular loops located between the transmembrane domains of CCR8. In yet another particular embodiment, the Treg depletor binds to one or more extracellular loops located between the transmembrane domains of CCR8. In another particular embodiments, the epitope(s) of the Treg depletor are located in said N-terminal region. In yet another embodiment, the epitope(s) of the Treg depletor are not located in the extracellular loops between the transmembrane domains.

In a further embodiment, the present invention provides nucleic acid molecules encoding a Treg depletor as defined herein. In some embodiments, such provided nucleic acid molecules may contain codon-optimized nucleic acid sequences. In another embodiment, the nucleic acid is included in an expression cassette within appropriate nucleic acid vectors for the expression in a host cell such as, for example, bacterial, yeast, insect, piscine, murine, simian, or human cells. In some embodiments, the present invention provides host cells comprising heterologous nucleic acid molecules (e.g. DNA vectors) that express the desired binder.

In some embodiments, the present invention provides methods of preparing an isolated Treg depletor as defined above. In some embodiments, such methods may comprise culturing a host cell that comprises nucleic acids (e.g. heterologous nucleic acids that may comprise and/or be delivered to the host cell via vectors). Preferably, the host cell (and/or the heterologous nucleic acid sequences) is/are arranged and constructed so that the Treg depletor is secreted from the host cell and isolated from cell culture supernatants.

### LTBR Agonist

As described herein, the term “LTBR agonist” refers to ligands specific for the receptor LTBR, which are compounds having the action of binding to the receptor, thus specifically stimulating ligand-dependent receptor activity (as differentiated from the baseline level determined in the absence of any ligand). This action is also simply referred to as a receptor-stimulating action or a receptor-activating action. Moreover, as synonyms for “agonist”, “activator”, “stimulator”, “receptor-activating ligand. Agonists include natural compounds, semisynthetic compounds derived from natural compounds, and synthetic compounds. LTBR agonists are known in the field and they are involved in the induction of high endothelial vesicles (HEVs) and tertiary lymphocyte structures (TLSs).

LTBR, also known as tumor necrosis factor receptor superfamily member 3 (TNFRSF3), is a cell surface receptor for lymphotoxin involved in apoptosis and cytokine release. It is a member of the tumor necrosis factor receptor superfamily. It is expressed on the surface of most cell types, including cells of epithelial and myeloid lineages, but not on T and B lymphocytes. The protein specifically binds the lymphotoxin membrane form (a complex of lymphotoxin-alpha and lymphtoxin-beta). The encoded protein and its ligand play a role in the development and organization of lymphoid tissue.

Lymphotoxin-alpha/beta/beta (Lymphotoxin-αββ) is a heterotrimeric species comprised of one subunit or copy of lymphotoxin-alpha and two subunits or copies of lymphotoxin-beta. Lymphotoxin-αββ binds to the lymphotoxin-beta receptor (LTBR). The activation of LTBR initiates a signaling event resulting in the expression of chemokines, including but not limited to, CXCL12, CXCL13, CCL19, and CCL21. These chemokines serve to induce the migration of dendritic cells, T-cells, and B-cells to establish the germinal center. Lymphotoxin-αββ is thus an LTBR agonist and HEV inducer suitable for application in the present invention.

LIGHT, also known as tumor necrosis factor superfamily member 14 (TNFSF14), is a member of the TNF superfamily, and its receptors have been identified as lymphotoxin beta receptor (LTBR), herpes virus entry mediator (HVEM), and decoy receptor 3 (DcR3). LIGHT stands for “homologous to lymphotoxin, exhibits inducible expression and competes with HSV glycoprotein D for binding to herpesvirus entry mediator, a receptor expressed on T lymphocytes”. In the cluster of differentiation terminology it is classified as CD258. This protein may function as a costimulatory factor for the activation of lymphoid cells. It is a known LTBR agonist and HEV inducer.

As will be understood by the skilled person, in principle any type of agonist of LTBR can be used in the present invention and different types of agonists are readily available to the skilled person or can be generated using the typical knowledge in the art, including small molecules and biologics or biologic-derived molecules. In a particular embodiment, the binding moiety of the LTBR agonist is proteinaceous, more particularly an LTBR agonistic polypeptide. In a further embodiment, the binding moiety of the LTBR agonist is antibody based or non-antibody based, preferably antibody based. Non-antibody based agonists include, but are not limited to, affibodies, Kunitz domain peptides, monobodies (adnectins), anticalins, designed ankyrin repeat domains (DARPins), centyrins, fynomers, avimers; affilins; affitins, peptides and the like.

In a particular embodiment, the LTBR agonist is selected from Lymphotoxin-αββ, LIGHT, or LTBR binding fragments or mimetics thereof. In another embodiment, the LTBR agonist comprises lymphotoxin alpha or lymphotoxin beta. In a further embodiment, the LTBR agonist is a fusion peptide comprising lymphotoxin alpha and lymphotoxin beta, in particular one lymphotoxin alpha part and two lymphotoxin beta parts. Such LTBR agonists are, for example, disclosed in WO2018119118 A1 and WO9622788 A1, which are incorporated herein by reference. In a particular embodiment, the LTBR agonist comprises SEQ ID NO: 16, SEQ ID NO: 17, or SEQ ID NO: 18 of WO2018119118 A1.

LIGHT and LIGHT mimetic peptides are also known in the art, e.g. from WO2018119118 A1. In certain embodiments, the LTBR agonist comprises LIGHT (e.g., human LIGHT) or a fragment thereof. As a non-limiting example, the LTBR-binding moiety may comprise the extracellular domain of LIGHT or a fragment thereof. In certain embodiments, the LTBR agonist comprises a LIGHT homotrimer (e.g., a single-chain LIGHT homotrimer). For instance, the LTBR agonist may comprise the extracellular domain of human LIGHT, a variant thereof having at least 80% sequence identity to the extracellular domain of human LIGHT, or a fragment thereof. In certain embodiments, the LTBR agonist may comprise a polypeptide (e.g., a LIGHT homotrimer) having at least about 80%, at least about 90%, at least about 95%, at least about 98%, or 100% sequence identity to SEQ ID NO:85 of WO2018119118 A1. In some embodiments, the LTBR agonist is a single-chain polypeptide. In certain embodiments, the LTBR agonist comprises a polypeptide having at least about 90%, at least about 95%, or at least about 98% sequence identity to SEQ ID NO:86 of WO2018119118 A1. For example, the LTBR agonist may comprise SEQ ID NO:86 of WO2018119118 A1. In some embodiments, the LTBR agonist comprises a mutant LIGHT homotrimer that has reduced the ability to bind to or activate HVEM.

In a particular embodiment, the LTBR agonist does not have cytotoxic activity. In a further embodiment, the LTBR agonist does not have ADCC, CDC or ADCP activity. In another embodiment, the LTBR agonist does not cause lysis of the cell it binds to. In another particular embodiment, the LTBR agonist does not deplete cells that it binds to.

In a preferred embodiment, the agonist comprises an LTBR agonistic moiety that is an antibody or active antibody fragment. In a further aspect of the invention, the agonist is an antibody (“agonistic antibody”). Agonistic antibodies that specifically bind LTBR are known in the art. For example, see WO2006/114284 A2, WO2004/058191 A2, and WO02/30986 A2, each of which is hereby incorporated by reference herein. In a further aspect of the invention the antibody is monoclonal. The antibody may additionally or alternatively be humanised or human. In a further aspect, the antibody is human, or in any case an antibody that has a format and features allowing its use and administration in human subjects. Antibodies may be derived from any species, including but not limited to mouse, rat, chicken, rabbit, goat, bovine, non-human primate, human, dromedary, camel, llama, alpaca, and shark.

In one aspect of the invention, the LTBR agonist comprises an active antibody fragment.

In another embodiment, the LTBR agonist as detailed above, comprises at least one single domain antibody moiety. Preferably, the LTBR agonist comprises at least two single domain antibody moieties.

In a further embodiment of the present invention, the LTBR agonist, as detailed above, comprises at least one Fc region moiety and at least two single domain antibody moieties that bind to LTBR. Preferably, the LTBR agonist is a genetically engineered polypeptide that comprises at least one Fc region moiety and at least two single domain antibody moieties that bind to LTBR, joined together by a peptide linker. The amino acid sequence of the Fc region moiety and/or the single domain antibody moiety region(s) may be humanized to reduce immunogenicity for humans.

In particular, the single domain antibody may be a Nanobody® (as defined herein) or a suitable fragment thereof (Note: Nanobody®, Nanobodies® and Nanoclone® are registered trademarks of Ablynx N.V., a Sanofi Company). Furthermore, as for full-size antibodies, single variable domains such as VHHs and Nanobodies® can be subjected to humanization and give humanized single domain antibodies. In another particular embodiment, the LTBR agonist does not comprise an Fc domain. In a further particular embodiment, the LTBR agonist comprises one or more single domain antibody moieties and does not comprise an Fc domain. Techniques for generating LTBR agonists are available to the person skilled in the art.

In a further embodiment, the present invention provides nucleic acid molecules encoding an LTBR agonist as defined herein. In some embodiments, such provided nucleic acid molecules may contain codon-optimized nucleic acid sequences. In another embodiment, the nucleic acid is included in an expression cassette within appropriate nucleic acid vectors for the expression in a host cell such as, for example, bacterial, yeast, insect, piscine, murine, simian, or human cells. In some embodiments, the present invention provides host cells comprising heterologous nucleic acid molecules (e.g. DNA vectors) that express the desired binder.

In some embodiments, the present invention provides methods of preparing an isolated LTBR agonist as defined above. In some embodiments, such methods may comprise culturing a host cell that comprises nucleic acids (e.g. heterologous nucleic acids that may comprise and/or be delivered to the host cell via vectors). Preferably, the host cell (and/or the heterologous nucleic acid sequences) is/are arranged and constructed so that the LTBR agonist is secreted from the host cell and isolated from cell culture supernatants.

### Combination

As mentioned above, the inventors have surprisingly observed a synergistic effect when the Treg depletor and the LTBR agonist as defined in the combination of the present invention are used. One object of the invention is thus a combination comprising a Treg depletor and an LTBR agonist. As will be understood from the disclosures made herein, the combination of the particular Treg depletors and the particular LTBR agonists described herein are objects of the invention. Further thereto, the combination of Treg depletors that are mentioned as being preferred embodiments with LTBR agonists that are mentioned as preferred embodiment, constitute preferred embodiments in relation to the combination, compositions comprising combinations and therapies relating to such combination.

In a preferred embodiment, the Treg depletor binds to a cell surface marker of a Treg cell and has cytotoxic activity.

In another preferred embodiment, the cell surface marker of the Treg cell is selected from the group consisting of CCR8, CCR4, CTLA4, CD25, TIGIT, OX40, ICOS, CD38, GITR, 4-1BB, NRP1, and LAG-3. In a particular embodiment, the cell surface marker of a Treg is selected from CCR8, CCR4, CD25, TIGIT, and ICOS; preferably CCR8, CD25, and CCR4.

In a more preferred embodiment, the cell surface marker of the Treg cell is CCR8. Therefore, in such a preferred embodiment, the Treg depletor is a CCR8 binder.

In a yet preferred embodiment, the cytotoxic activity of the Treg depletor, in particular of the CCR8 binder, is caused by the presence of a cytotoxic moiety that induces antibody-dependent cellular cytotoxicity (ADCC), induces complement-dependent cytotoxicity (CDC), induces antibody-dependent cellular phagocytosis (ADCP), binds to and activates T-cells, or comprises a cytotoxic payload.

Preferably, the cytotoxic moiety comprises a fragment crystallisable (Fc) region moiety.

Advantageously, the Fc region moiety has been engineered to increase ADCC, CDC, and/or ADCP activity, such as through afucosylation or by comprising an ADCC, CDC and/or ADCP-increasing mutation.

In a yet preferred embodiment, the Treg depletor, in particular the CCR8 binder, comprises at least one single domain antibody moiety that binds to a cell surface marker of Treg, in particular to CCR8.

In a particular embodiment, the combination of the present invention comprises a marker binding antibody also referenced herein as to “Treg depleting antibody”, in particular a CCR8 binding antibody, with ADCC, CDC and/or ADCP activity and an LTBR agonistic antibody. Therefore, in a particular embodiment, both the Treg depletor and the LTBR agonist are an antibody, in particular a distinct antibody. In a preferred embodiment the Treg depletor is an antibody that binds to CCR8, CCR4, CTLA4, CD25, TIGIT, OX40, ICOS, CD38, GITR, 4-1BB, NRP1, and LAG-3 and the LTBR agonist is an LTBR binding agonistic antibody. In a further particular embodiment, the Treg depletor is a CCR8 binding antibody and the LTBR agonist is an LTBR binding agonistic antibody.

In another embodiment, the combination of the present invention further comprises one or more pharmaceutically acceptable carriers or excipients of it. In one embodiment, said one or more pharmaceutically acceptable carriers or excipients of it can be present with the Treg depletor, in particular with the CCR8 binder, and/or the LTBR agonist. Thus, the combination of the invention can either comprises a first composition comprising the Treg depletor, in particular the CCR8 binder, with said one or more pharmaceutically acceptable carriers or excipients of it and the LTBR agonist; or comprises the Treg depletor, in particular the CCR8 binder, and a second composition comprising the LTBR agonist with said one or more pharmaceutically acceptable carriers or excipients of it; or comprises said first and second compositions i.e. the Treg depletor, in particular the CCR8 binder, with said one or more pharmaceutically acceptable carriers or excipients of it and the LTBR agonist with said one or more pharmaceutically acceptable carriers or excipients of it.

Combination as used herein refers to a combination of two features (Treg depletion and LTBR agonism). These features may be present in a single molecules, e.g. a molecule comprising a Treg binding portion and an LTBR agonizing portion. Although bispecific antibodies are a possibility for performing the present invention, as will be described herein below, in a particular and preferred embodiment, the Treg depletor and LTBR agonist for use in the invention are distinct molecules. In a more particular embodiment, the Treg depletor is an antibody, such as a cytotoxic CCR8 binding antibody, as described herein and the LTBR agonist is a distinct molecule, preferably and LTBR agonistic antibody. In a further preferred embodiment, the LTBR agonist does not comprise a cytotoxic moiety as defined herein.

### Composition

Another object of the invention is a composition comprising the combination of the present invention. Thus, the composition of the invention comprises a Treg depletor, in particular a CCR8 binder, binding to a cell surface marker of a Treg, in particular to CCR8, and having cytotoxic activity and an LTBR agonist.

In a preferred embodiment, the composition of the invention comprises a marker binding antibody also referenced herein as to “Treg depleting antibody”, in particular a CCR8 binding antibody, with ADCC, CDC and/or ADCP activity and an LTBR agonistic antibody.

In a yet preferred embodiment, the composition of the invention further comprises one or more pharmaceutically acceptable carriers or excipients of it.

### Bispecific Molecule

Yet another aspect of the invention is a bispecific molecule comprising a Treg depleting moiety, in particular a CCR8 binding moiety, and an LTBR agonistic moiety, wherein the bispecific molecule has cytotoxic activity.

As used herein, “bispecific” refers to a molecule having the capacity to bind two distinct epitopes on two different antigens or polypeptides, one of which being an LTBR antigen or polypeptide.

In a preferred embodiment, the cytotoxic activity of the bispecific molecule is caused by the Treg depleting moiety, in particular by the CCR8 binding moiety, that induces antibody-dependent cellular cytotoxicity (ADCC), induces complement-dependent cytotoxicity (CDC), induces antibody-dependent cellular phagocytosis (ADCP), binds to and activates T-cells, or comprises a cytotoxic payload.

In a particular embodiment, the Treg depleting moiety, in particular the CCR8 binding moiety, is proteinaceous, more particularly a Treg depleting polypeptide (i.e. a marker binding polypeptide), in particular a CCR8 binding polypeptide. In a further embodiment, the Treg depleting moiety, in particular the CCR8 binding moiety, is antibody based or non-antibody based, preferably antibody based. In a preferred embodiment, the Treg depleting moiety, in particular the CCR8 binding moiety, is an antibody or active antibody fragment.

In another embodiment, the Treg depleting moiety, in particular the CCR8 binding moiety, comprises at least one single domain antibody moiety. Preferably, the Treg depleting moiety, in particular the CCR8 binding moiety comprises at least two single domain antibody moieties.

In a further embodiment, the cytotoxic moiety comprises an antibody or antigen-binding fragment thereof that binds to CD3. Thus, the Treg depleting moiety, in particular the CCR8 binding moiety, may bind to a cell surface marker of Treg, in particular to CCR8, and CD3. Such a Treg depletor binds to intratumoural Tregs and directs the cytotoxic activity of T-cells to these Tregs, thereby depleting them from the tumour environment. In a particular embodiment, the Treg depletor of the invention comprises a moiety that binds to a cell surface marker of Treg, in particular to CCR8, and a moiety that binds to CD3, wherein at least one moiety is antibody based, particularly wherein both moieties are antibody based. Therefore, in a particular embodiment, the present invention provides a bispecific construct comprising an antibody or antigen-binding fragment thereof that specifically binds to a cell surface marker of Treg, in particular to CCR8, and an antibody or antigen-binding fragment thereof that specifically binds to CD3.

In one embodiment, the cytotoxic moiety comprises a fragment crystallisable (Fc) region moiety. Within the context of the present invention the term “fragment crystallisable (Fc) region moiety” refers to the crystallisable fragment of an immunoglobulin molecule composed of the constant regions of the heavy chains and responsible for the binding to antibody Fc receptors and some other proteins of the complement system, thereby inducing ADCC, CDC, and/or ADCP activity.

In a further embodiment of the present invention, the Treg depleting moiety, in particular the CCR8 binding moiety, comprises at least one Fc region moiety and at least two single domain antibody moieties that bind to a cell surface marker of Treg, in particular to CCR8. Preferably, the Treg depleting moiety, in particular the CCR8 binding moiety, is a genetically engineered polypeptide that comprises at least one Fc region moiety and at least two single domain antibody moieties that bind to a cell surface marker of a Treg, in particular to CCR8, joined together by a peptide linker. The amino acid sequence of the Fc region moiety and/or the single domain antibody moiety region(s) may be humanized to reduce immunogenicity for humans.

In one embodiment, the Fc region moiety has been engineered to increase ADCC, CDC and/or ADCP activity.

In a particular embodiment of the present invention, the Treg depleting moiety, in particular the CCR8 binding moiety, comprising an Fc region moiety is optimized to elicit an ADCC response, that is to say the ADCC response is enhanced, increased or improved relative to other ones, in particular to other CCR8 binders, comprising an Fc region moiety, including those that do not inhibit the binding of a ligand, in particular of CCL1, to its cell surface marker of Tregs, in particular to CCR8. In a preferred embodiment, the Treg depletor, in particular the CCR8 binder, has been engineered to elicit an enhanced ADCC response.

In a preferred embodiment of the present invention, the Treg depletor, in particular the CCR8 binder, comprising an Fc region moiety is optimized to elicit an ADCP response, that is to say the ADCP response is enhanced, increased or improved relative to other ones, in particular to other ones, in particular to other CCR8 binders, comprising an Fc region moiety, including those that do not inhibit the binding of a ligand, in particular of CCL1, to its receptor (cell surface marker), in particular to CCR8.

In another embodiment, the cytotoxic moiety comprises a moiety that binds to an Fc gamma receptor. More in particular binds to and activates an FcγR, in particular an activating receptor, such as FcγRI and/or FcγRIIa and/or FcγRIIIa, especially FcγRIIIa. The moiety that binds to an FcγR may be antibody based or non-antibody based as described herein before. If antibody based, the moiety may bind the FcγR through its variable region.

The bispecific molecule of the present invention as discussed herein can be produced via biological methods, such as somatic hybridization; or genetic methods, such as the expression of a non-native DNA sequence encoding the desired binder structure in a cell line or in an organism; chemical methods (e.g. by chemical coupling, genetic fusion, noncovalent associated or otherwise to one or more molecular entities, such as another binder of fragment thereof); or combination thereof.

The technologies and products that allow producing bispecific molecules are known in the art, as extensively reviewed in the literature, also with respect to alternative formats, Treg depletor-drug conjugates, Treg depletor design methods, in vitro screening methods, constant regions, post-translational and chemical modifications, improved feature for triggering cancer cell death such as Fc domain engineering (Tiller K and Tessier P, Annu Rev Biomed Eng. 17:191-216 (2015); Speiss C et al., Molecular Immunology 67:95-106 (2015); Weiner G, Nat Rev Cancer, 15:361-370 (2015); Fan G et al., J Hematol Oncol 8:130 (2015)).

In a further embodiment, the present invention provides a nucleic acid molecule encoding the bispecific molecule as defined herein. In some embodiments, such provided nucleic acid molecule may contain codon-optimized nucleic acid sequences. In another embodiment, the nucleic acid is included in an expression cassette within appropriate nucleic acid vectors for the expression in a host cell such as, for example, bacterial, yeast, insect, piscine, murine, simian, or human cells. In some embodiments, the present invention provides host cells comprising heterologous nucleic acid molecules (e.g. DNA vectors) that express the desired binder.

In a particular embodiment, the bispecific molecule of the invention is administered as a therapeutic nucleic acid. The term “therapeutic nucleic acid” used herein refers to any nucleic acid molecule that have a therapeutic effect when introduced into a eukaryotic organism (e.g., a mammal such as human) and includes DNA and RNA molecules encoding the binder of the invention. As is known to the skilled person, the nucleic acid may comprise elements that induce transcription and/or translation of the nucleic acid or that increases ex and/or in vivo stability of the nucleic acid.

### Treatment

A further object of the invention is a combination presenting the features as described herein, a composition comprising such a combination, a bispecific molecule presenting the features as described herein, as well as a nucleic acid encoding such a bispecific molecule, for use as a medicine.

Another object of the invention is a combination presenting the features as described herein, a composition comprising such a combination, a bispecific molecule presenting the features as described herein, as well as a nucleic acid encoding such a bispecific molecule, for use in the treatment of a cancer.

Yet another object of the invention is a Treg depletor, in particular a CCR8 binder, presenting the features as described herein for use in the treatment of a cancer, wherein the treatment further comprises the administration of an LTBR agonist presenting the features as described herein.

Preferably, the Treg depletor, in particular the CCR8 binder, is a Treg depleting antibody, in particular a CCR8 binding antibody, that binds to a cell surface marker of a Treg, in particular to CCR8, and that has ADCC, CDC and/or ADCP activity; and the LTBR agonist is an LTBR agonistic antibody.

Still another object of the invention is an LTBR agonist presenting the features as described herein for use in the treatment of a cancer, wherein the treatment further comprises the administration of a Treg depletor, in particular a CCR8 binder, presenting the features as described herein.

In a further embodiment the invention provides a method for treating a disease in a subject comprising administering the combination of the present invention, the composition comprising such a combination, the bispecific molecule of the present invention, as well as the nucleic acid encoding such a bispecific molecule. Preferably the disease is a cancer, in particular the treatment of solid tumours.

In a further embodiment the invention provides a method for treating a disease in a subject comprising the steps of:
administering the Treg depletor as defined herein; and
administering the LTBR agonist as defined herein, wherein both administrations are done separately, simultaneously or sequentially.

In another particular embodiment, the invention provides a method for treating a disease in a subject undergoing Treg depletion therapy, the method comprising administering an LTRB agonist to said subject.

Preferably the disease is a cancer, in particular the treatment of solid tumours.

In a preferred embodiment of the present invention, the subject of the aspects of the invention as described herein, is a mammal, preferably a cat, dog, horse, donkey, sheep, pig, goat, cow, hamster, mouse, rat, rabbit, or guinea pig, but most preferably the subject is a human. Thus in all aspects of the invention as described herein the subject is preferably a human.

As used herein, the terms “cancer”, “cancerous”, or “malignant” refer to or describe the physiological condition on mammals that is typically characterized by unregulated cell growth.

As used herein, the term “tumour” as it applies to a subject diagnosed with, or suspected of having, a cancer refers to a malignant or potentially malignant neoplasm or tissue mass of any size, and includes primary tumours and secondary neoplasms. The terms “cancer”, “malignancy”, “neoplasm”, “tumour” and “carcinoma” can also be used interchangeably herein to refer to tumours and tumour cells that exhibit an aberrant growth phenotype characterized by a significant loss of control of cell proliferation. In general, cells of interest for treatment include precancerous (e.g. benign), malignant, pre-metastatic, metastatic, and non-metastatic cells. The teachings of the present disclosure may be relevant to any and all tumours.

Examples of tumours include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hepatocellular carcinoma (HCC), hodgkin's lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer.

In one aspect, the tumour involves a solid tumour. Examples of solid tumours are sarcomas (including cancers arising from transformed cells of mesenchymal origin in tissues such as cancellous bone, cartilage, fat, muscle, vascular, hematopoietic, or fibrous connective tissues), carcinomas (including tumours arising from epithelial cells), mesothelioma, neuroblastoma, retinoblastoma, etc. Tumours involving solid tumours include, without limitations, brain cancer, lung cancer, stomach cancer, duodenal cancer, esophagus cancer, breast cancer, colon and rectal cancer, renal cancer, bladder cancer, kidney cancer, pancreatic cancer, prostate cancer, ovarian cancer, melanoma, mouth cancer, sarcoma, eye cancer, thyroid cancer, urethral cancer, vaginal cancer, neck cancer, lymphoma, and the like.

In another particular embodiment, the tumour is selected from the group consisting of breast invasive carcinoma, colon adenocarcinoma, head and neck squamous carcinoma, stomach adenocarcinoma, lung adenocarcinoma (NSCLC), lung squamous cell carcinoma (NSCLC), kidney renal clear cell carcinoma, skin cutaneous melanoma, esophageal cancer, cervical cancer, hepatocellular carcinoma, merkel cell carcinoma, small Cell Lung Cancer (SCLC), classical Hodgkin Lymphoma (cHL), urothelial Carcinoma, Microsatellite Instability-High (MSI-H) Cancer and mismatch repair deficient (dMMR) cancer.

In a further embodiment, the tumour is selected from the group consisting of a breast cancer, uterine corpus cancer, lung cancer, stomach cancer, head and neck squamous cell carcinoma, skin cancer, colorectal cancer, and kidney cancer. In an even further embodiment, the tumour is selected from the group consisting of breast invasive carcinoma, colon adenocarcinoma, head and neck squamous carcinoma, stomach adenocarcinoma, lung adenocarcinoma (NSCLC), lung squamous cell carcinoma (NSCLC), kidney renal clear cell carcinoma, and skin cutaneous melanoma. In one aspect, the cancers involve CCR8 expressing tumours, including but not limited to breast cancer, uterine corpus cancer, lung cancer, stomach cancer, head and neck squamous cell carcinoma, skin cancer, colorectal cancer, and kidney cancer. In one particular embodiment, the tumour is selected from the group consisting of breast cancer, colon adenocarcinoma, and lung carcinoma.

As used herein, the term “administration” refers to the act of giving a drug, prodrug, antibody, or other agent, or therapeutic treatment to a physiological system (e.g. a subject or in vivo, in vitro, or ex vivo cells, tissues, and organs). Exemplary routes of administration to the human body can be through the mouth (oral), skin (transdermal), oral mucosa (buccal), ear, by injection (e.g. intravenously, subcutaneously, intratumourally, intraperitoneally, etc.) and the like. The term administration of the Treg depletor or of the LTBR agonist of the invention includes direct administration of the Treg depletor or of the LTBR agonist as well as indirect administration by administering a nucleic acid encoding the Treg depletor or the LTBR agonist, such that the Treg depletor or the LTBR agonist is produced from the nucleic acid in the subject. Administration of the Treg depletor or of the LTBR agonist thus includes DNA and RNA therapy methods that result in in vivo production of the Treg depletor or the LTBR agonist.

Reference to “treat” or “treating” a tumour as used herein defines the achievement of at least one therapeutic effect, such as for example, reduced number of tumour cells, reduced tumour size, reduced rate to cancer cell infiltration into peripheral organs, or reduced rate of tumour metastasis or tumour growth. As used herein, the term “modulate” refers to the activity of a compound to affect (e.g. to promote or treated) an aspect of the cellular function including, but not limited to, cell growth, proliferation, invasion, angiogenesis, apoptosis, and the like.

Positive therapeutic effects in cancer can be measured in a number of ways (e.g. Weber (2009) J Nucl Med 50, 1S-10S). By way of example, with respect to tumour growth inhibition, according to National Cancer Institute (NCI) standards, a T/C≤42% is the minimum level of anti-tumour activity. A T/C<10% is considered a high anti-tumour activity level, with T/C (%)=Median tumour volume of the treated/Median tumour volume of the control×100. In some embodiments, the treatment achieved by a therapeutically effective amount is any of progression free survival (PFS), disease free survival (DFS) or overall survival (OS). PFS, also referred to as “Time to Tumour Progression” indicates the length of time during and after treatment that the cancer does not grow, and includes the amount of time patients have experienced a complete response or a partial response, as well as the amount of time patients have experienced stable disease. DFS refers to the length of time during and after treatment that the patient remains free of disease. OS refers to a prolongation in life expectancy as compared to naive or untreated individuals or patients.

Reference to “prevention” (or prophylaxis) as used herein refers to delaying or preventing the onset of the symptoms of the cancer. Prevention may be absolute (such that no disease occurs) or may be effective only in some individuals or for a limited amount of time.

In a preferred aspect of the invention the subject has an established tumour that is the subject already has a tumour e.g. that is classified as a solid tumour. As such, the invention as described herein can be used when the subject already has a tumour, such as a solid tumour. As such, the invention provides a therapeutic option that can be used to treat an existing tumour. In one aspect of the invention the subject has an existing solid tumour. The invention may be used as a prevention, or preferably as a treatment in subjects who already have a solid tumour. In one aspect the invention is not used as a preventative or prophylaxis.

In one aspect, tumour regression may be enhanced, tumour growth may be impaired or reduced, and/or survival time may be enhanced using the invention as described herein, for example compared with other cancer treatments (for example standard-of care treatments for the a given cancer).

In one aspect of the invention the method of treatment or prevention of a tumour as described herein further comprises the step of identifying a subject who has tumour, preferably identifying a subject who has a solid tumour.

The dosage regimen of a therapy described herein that is effective to treat a patient having a tumour may vary according to factors such as the disease state, age, and weight of the patient, and the ability of the therapy to elicit an anti-cancer response in the subject. Selection of an appropriate dosage will be within the capability of one skilled in the art. For example 0.01, 0.1, 0.3, 0.5, 1, 2, 3, 4, 5, 6, 7, 8, 9, 10, 20, 30, 40, or 50 mg/kg. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen).

The combination, the composition and the bispecific molecule according to any aspect of the invention as described herein, may be in the form of a pharmaceutical composition which additionally comprises a pharmaceutically acceptable carrier, diluent or excipient. As used herein, the term “pharmaceutically acceptable carrier” or “pharmaceutically acceptable excipient” includes any material which, when combined with an active ingredient, allows the ingredient to retain biological activity. Pharmaceutically acceptable carriers enhance or stabilize the composition or can be used to facilitate preparation of the composition. Pharmaceutically acceptable carriers include solvents, dispersion media, coatings, antibacterial and antifungal agents, isotonic and absorption delaying agents, and the like that are physiologically compatible, as is known to those skilled in the art (see, for example, Remington's Pharmaceutical Sciences, 18th Ed. Mack Printing Company, 1990, pp. 1289-1329; Remington: The Science and Practice of Pharmacy, 21st Ed. Pharmaceutical Press 2011; and subsequent versions thereof). Non-limiting examples of said pharmaceutically acceptable carrier comprise any of the standard pharmaceutical carriers such as a phosphate buffered saline solution, water, emulsions such as oil/water emulsion, and various types of wetting agents.

These compositions include, for example, liquid, semi-solid and solid dosage formulations, such as liquid solutions (e.g., injectable and infusible solutions), dispersions or suspensions, tablets, pills, or liposomes. In some embodiments, a preferred form may depend on the intended mode of administration and/or therapeutic application. Pharmaceutical compositions containing the combination, the composition or the bispecific molecule can be administered by any appropriate method known in the art, including, without limitation, oral, mucosal, by-inhalation, topical, buccal, nasal, rectal, or parenteral (e.g. intravenous, infusion, intratumoural, intranodal, subcutaneous, intraperitoneal, intramuscular, intradermal, transdermal, or other kinds of administration involving physical breaching of a tissue of a subject and administration of the pharmaceutical composition through the breach in the tissue). Such a formulation may, for example, be in a form of an injectable or infusible solution that is suitable for intradermal, intratumoural or subcutaneous administration, or for intravenous infusion. In a particular embodiment, the binder or nucleic acid is administered intravenously. The administration may involve intermittent dosing. Alternatively, administration may involve continuous dosing (e.g., perfusion) for at least a selected period of time, simultaneously or between the administration of other compounds.

Formulations of the invention generally comprise therapeutically effective amounts of the treg depletor, in particular the CCR8 binder, and the LTBR agonist as defined in the combination of the invention. “Therapeutic levels”, “therapeutically effective amount” or “therapeutic amount” means an amount or a concentration of an active agent that has been administered that is appropriate to safely treat the condition to reduce or prevent a symptom of the condition.

In some embodiments, the Treg depletor, in particular the CCR8 binder and the LTBR agonist as defined in the combination of the present invention can be prepared with carriers that protect it against rapid release and/or degradation, such as a controlled release formulation, such as implants, transdermal patches, and microencapsulated delivery systems. Biodegradable, biocompatible polymers can be used.

Those skilled in the art will appreciate, for example, that route of delivery (e.g., oral vs intravenous vs subcutaneous vs intratumoural, etc) may impact dose amount and/or required dose amount may impact route of delivery. For example, where particularly high concentrations of an agent within a particular site or location (e.g., within a tumour) are of interest, focused delivery (e.g., in this example, intratumoural delivery) may be desired and/or useful. Other factors to be considered when optimizing routes and/or dosing schedule for a given therapeutic regimen may include, for example, the particular cancer being treated (e.g., type, stage, location, etc.), the clinical condition of a subject (e.g., age, overall health, etc.), the presence or absence of combination therapy, and other factors known to medical practitioners. In a particular embodiment, the Treg depletor is administered intravenously. In another particular embodiment, the LTBR agonist is administered intravenously. In a further particular embodiment, the Treg depletor and the LTBR agonist are administered intravenously.

The pharmaceutical compositions typically should be sterile and stable under the conditions of manufacture and storage. The composition can be formulated as a solution, microemulsion, dispersion, liposome, or other ordered structure suitable to high drug concentration. Sterile injectable solutions can be prepared by incorporating the binder in the required amount in an appropriate solvent with one or a combination of ingredients enumerated above, as required, followed by filtered sterilization. Formulations for parenteral administration include, but are not limited to, suspensions, solutions, emulsions in oily or aqueous vehicles, pastes, and implantable sustained-release or biodegradable formulations as discussed herein. Sterile injectable formulations may be prepared using a non-toxic parenterally acceptable diluent or solvent. Each pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers are non-toxic to the subjects at the dosages and concentrations employed. Preferably, such a composition can further comprise a pharmaceutically acceptable carrier or excipient for use in the treatment of cancer that that is compatible with a given method and/or site of administration, for instance for parenteral (e.g. subcutaneous, intradermal, or intravenous injection), intratumoural, or peritumoural administration.

While an embodiment of the treatment method or compositions for use according to the present invention may not be effective in achieving a positive therapeutic effect in every subject, it should do so in a using pharmaceutical compositions and dosing regimens that are consistently with good medical practice and statistically significant number of subjects as determined by any statistical test known in the art such as the Student's t-test, the X²-test, the U-test according to Mann and Whitney, the Kruskal-Wallis test (H-test), Jonckheere-Terpstra test and the Wilcoxon-test.

Where hereinbefore and subsequently a tumour, a tumour disease, a carcinoma or a cancer is mentioned, also metastasis in the original organ or tissue and/or in any other location are implied alternatively or in addition, whatever the location of the tumour and/or metastasis is.

In some embodiments, a different agent against cancer may be administered in combination with the combination, the composition or the bispecific molecule of the invention via the same or different routes of delivery and/or according to different schedules. Alternatively or additionally, in some embodiments, one or more doses of a first active agent is administered substantially simultaneously with, and in some embodiments via a common route and/or as part of a single composition with, one or more other active agents. Those skilled in the art will further appreciate that some embodiments of combination therapies provided in accordance with the present invention achieve synergistic effects; in some such embodiments, dose of one or more agents utilized in the combination may be materially different (e.g., lower) and/or may be delivered by an alternative route, than is standard, preferred, or necessary when that agent is utilized in a different therapeutic regimen (e.g., as monotherapy and/or as part of a different combination therapy).

In some embodiments, where two or more active agents are utilized in accordance with the present invention, such agents can be administered simultaneously or sequentially. In some embodiments, administration of one agent is specifically timed relative to administration of another agent. For example, in some embodiments, a first agent is administered so that a particular effect is observed (or expected to be observed, for example based on population studies showing a correlation between a given dosing regimen and the particular effect of interest). In some embodiments, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular patient of interest.

“In combination” or treatments comprising administration of a further therapeutic may refer to administration of the additional therapy before, at the same time as or after administration of any aspect according to the present invention. Combination treatments can thus be administered simultaneous, separate or sequential.

In another embodiment, the invention provides a kit comprising the combination, the composition and/or the bispecific molecule described above. In some embodiments, the kit further contains a pharmaceutically acceptable carrier or excipient of it. In other related embodiments, any of the components of the above combinations in the kit are present in a unit dose, in particular the dosages as described herein. In a yet further embodiment, the kit includes instructions for use in administering any of the components or the above combinations to a subject. In one particular embodiment, the kit comprises a Treg depletor, in particular a CCR8 binder, as described herein and an LTBR agonist. The Treg depletor, in particular the CCR8 binder and the LTBR agonsit can be present in the same or in a different composition.

In one particular embodiment, the present invention provides a package comprising a combination, a composition and/or a bispecific molecule as described herein, wherein the package further comprises a leaflet with instructions to administer the binder to a tumour patient that also receives treatment with an immune checkpoint inhibitor.

In yet another particular embodiment, the present invention provides the use of an LTBR agonist for the manufacture of a medicament for the treatment of a disease as described herein, wherein the treatment further comprises administration of a Treg depletor as described herein. In another particular embodiment, the present invention provides the use of a Treg depletor as described herein for the manufacture of a medicament for the treatment of a disease as described herein, wherein the treatment further comprises administration of an LTBR agonist. In another further embodiment, the present invention provides the use of an LTRB agonist and a Treg depletor as described herein for the manufacture of a medicament for the treatment of a disease as described herein. The present invention further provides pharmaceutical compositions as described herein for the treatment of a disease as described herein, particularly cancer.

The invention will now be further described by way of the following Example, which are meant to serve to assist one of ordinary skill in the art in carrying out the invention and are not intended in any way to limit the scope of the invention, with reference to the drawings.

### EXAMPLES

The following examples are provided in order to demonstrate and further illustrate certain preferred embodiments and aspects of the present invention and are not construed as limiting the scope thereof.

### Example 1: LTBR Reporter Assay

### Generation of Stable LTBR Reporter Cell Line

A transgenic constructs was generated, carrying a mouse-human chimera LTBR coding sequence in which the intracellular part of the mouse orthologue was replaced by the human counterpart to ensure functional signaling in a human cell line background. A human NFκB Luciferase Reporter HEK293 stable cell line (Signosis, cat. #SL-0012) was cultured at 37° C. and 5% CO2 in Dulbecco's Modified Eagle Medium (DMEM, Gibco) supplemented with 10% heat-inactivated fetal bovine serum (FBS) and 100 U/mL penicillin and streptomycin (Gibco). Before transfection, cells were seeded at a density of 7.5×10⁵ cells per well of 6-well plates (Greiner) and cultured overnight. Upon reaching an approximate confluence of 40%, cells were transfected with linearized pcDNA3.1 carrying the mouse-human chimera LTBR transgene, using FUGENE HD transfection reagent (Promega). After 6 hours, cellular supernatants were carefully removed and replaced by fresh complete DMEM. After 48 hours, culture medium was replaced to include 500 μg/mL G-418 (Thermofisher Scientific) to select for geneticin-resistant transfectants harboring the expression cassette. Medium was changed every 2-3 days and after 3 weeks, limiting 1:2 dilutions were made starting from 10³ cells per well to obtain monoclonal lines. Identification of LTBR-expressing monoclonal lines was based on acquiring 10⁴ cells in flow cytometry (Attune NxT, Thermofisher Scientific) using a phycoerythrin-labelled mouse anti-mouse LTBR mAb 5G11 (Abcam, cat. #ab65089).

### Reporter Assay

Cells were plated in Poly-D-Lysine (PDL) coated 96-well plates (Greiner) at a density of 6.0×10⁴ cells/well and cultured overnight at 37° C. and 5% CO₂ in Dulbecco's Modified Eagle Medium (DMEM, Gibco) supplemented with 10% heat-inactivated fetal bovine serum (FBS) and 100 U/mL penicillin and streptomycin (Gibco). Compounds (VHHs and mAbs) were incubated at different concentrations for 6 hours to evaluate their agonistic activity on LTBR to induce NFκB transcription. Luciferase activity was measured using the Steadylite plus Reporter Gene Assay System (PerkinElmer, cat. #6066756) according to the manufacterer's instructions, on an EnSight™ Multimode Plate Reader (PerkinElmer). Final QC of the stable reporter cell line was done by means of a titration of the agonistic anti-mouse LTBR mAb 5G11 (Abcam, cat. #ab65089) which activates the reporter in a dose-dependent manner.

### Example 2: Generation of Mouse CCR8 VHH

### CCR8 DNA Immunization

Immunization of llamas and alpacas with CCR8 DNA was performed essentially as disclosed in Pardon E., et al. (A general protocol for the generation of Nanobodies for structural biology, Nature Protocols, 2014, 9(3), 674-693) and Henry K. A. and MacKenzie C. R. eds. (Single-Domain Antibodies: Biology, Engineering and Emerging Applications. Lausanne: Frontiers Media). Briefly, animals were immunized four times at two week intervals with 2 mg of DNA encoding mouse CCR8 inserted into the expression vector pVAX1 (ThermoFisher Scientific Inc., V26020), after which blood samples were taken. Three months later, all animals received a single administration of 2 mg the same DNA, after which blood samples were taken.

### Phage Display Library Preparation

Phage display libraries derived from peripheral blood mononuclear cells (PBMCs) were prepared and used as described in Pardon E., et al. (A general protocol for the generation of Nanobodies for structural biology, Nature Protocols, 2014, 9(3), 674-693) and Henry K. A. and MacKenzie C. R. eds. (Single-Domain Antibodies: Biology, Engineering and Emerging Applications. Lausanne: Frontiers Media). The VHH fragments were inserted into a M13 phagemid vector containing MYC and His6 tags. The libraries were rescued by infecting exponentially-growing Escherichia coli TG1 [(F′ tra D36 proAB laclqZ ΔM15) supE thi-1 Δ(lac-proAB) Δ(mcrB-hsdSM)5(rK−mK−)] cells followed by surinfection with VCSM13 helper phage.

Phage display libraries were subjected to two consecutive selection rounds on HEK293T cells transiently transfected with mouse CCR8 inserted into pVAX1 followed by CHO-K1 cells transiently transfected with mouse CCR8 inserted into pVAX1. Polyclonal phagemid DNA was prepared from E. coli TG1 cells infected with the eluted phages from the second selection rounds. The VHH fragments were amplified by means of PCR from these samples and subcloned into an E. coli expression vector, in frame with N-terminal PeIB signal peptide and C-terminal FLAG3 and His6 tags. Electrocompetent E. coli TG1 cells were transformed with the resulting VHH-expression plasmid ligation mixture and individual colonies were grown in 96-deep-well plates. Monoclonal VHHs were expressed essentially as described in Pardon E., et al. (A general protocol for the generation of Nanobodies for structural biology, Nature Protocols, 2014, 9(3), 674-693). The crude periplasmic extracts containing the VHHs were prepared by freezing the bacterial pellets overnight followed by resuspension in PBS and centrifugation to remove cellular debris.

### Screening for CCR8 Selection Outputs

Recombinant cells expressing CCR8 were recovered using cell dissociated non-enzymatic solution (Sigma Aldrich, C5914-100 mL) and resuspended to a final concentration of 1.0×10⁶ cells/ml in FACS buffer. Dilutions (1:5 in FACS buffer) of crude periplasmic extracts containing VHHs were incubated with mouse anti-FLAG biotinylated antibody (Sigma Aldrich, F9291-1MG) at 5 μg/ml in FACS buffer for 30 min with shaking at room temperature. Cell suspensions were distributed into 96-well v-bottom plates and incubated with the VHH/antibody mixture with one hour with shaking on ice. Binding of VHHs to cells was detected with streptavidin R-PE (Invitrogen, SA10044) at 1:400 dilution (0.18 μg/ml) in FACS buffer, incubated for 30 minutes in the dark with shaking on ice. Surface expression of mCCR8 on transiently transfected cell lines was confirmed by means of PE anti-mouse CCR8 (Biolegend, 150311) antibody at 2 μg/ml.

VHH clones resulting from the mouse CCR8 immunization and selection campaign were screened by means of flow cytometry for binding to HEK293 cells previously transfected with mCCR8 or with N-terminal deletion mouse CCR8 (deltal6-3XHA) plasmid DNA, in comparison to mock-transfected control cells. Comparison of the binding (median fluorescent intensity) signal of a given VHH clone across the three cell lines enabled classification of said clone as an N-terminal mouse CCR8 binder (i.e. binding on mCCR8 cells, but not on mouse CCR8 (deltal6-3XHA) or control cells) or as an extracellular loop mCCR8 binder (i.e. binding on mCCR8 cells and on mouse CCR8 (deltal6-3XHA), but not on control cells).

### Purification and Evaluation of Monovalent CCR8 VHHs

Synthetic DNA fragments encoding CCR8-binding VHHs were subcloned into an E. coli expression vector under control of an IPTG-inducible lac promoter, infra me with N-terminal PeIB signal peptide for periplasmic compartment-targeting and C-terminal FLAG3 and His6 tags. Electrocompetent E. coli TG1 cells were transformed and the resulting clones were sequenced. VHH proteins were purified from these clones by IMAC chromatography followed by desalting, essentially as described in Pardon E., et al. (A general protocol for the generation of Nanobodies for structural biology, Nature Protocols, 2014, 9(3), 674-693).

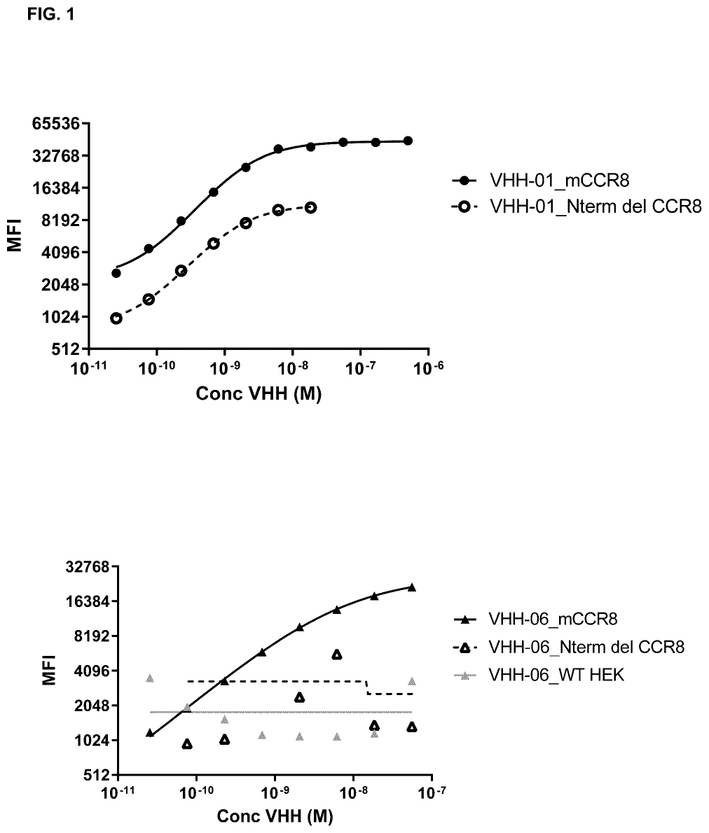

Two purified VHHs (VHH-01 and VHH-06, herein after) obtained from the mouse CCR8 immunization campaign were selected and evaluated by flow cytometry for their binding to mCCR8 as compared with N-terminal deletion mCCR8. The results of this assessment are summarized in FIG. 1. VHH-01 binds to both full-length and N-terminal deletion mouse CCR8 whereas VHH-06 only binds to full-length mouse CCR8.

### Binding and Functional Characterization for Monovalent CCR8 VHHs

### cAMP Homogenous Time Resolved Fluorescence (HTRF) Assay

The two selected monovalent VHHs (VHH-01 and VHH-06) were evaluated for their potential to functionally inhibit mouse CCL1 signalling on CHO-K1 cells displaying mouse CCR8 in cAMP accumulation experiments.

CHO-K1 cells stably expressing recombinant mouse CCR8 were grown prior to the test in media without antibiotic and detached by flushing with PBS-EDTA (5 mM EDTA), recovered by centrifugation and resuspended in KHR buffer (5 mM KCI, 1.25 mM MgSO₄, 124 mM NaCl, 25 mM HEPES, 13.3 mM Gluclose, 1.25 mM KH₂PO₄, 1.45 mM CaCl₂, 0.5 g/l BSA, supplemented with 1 mM IBMX). Twelve microliters of cells were mixed with six microliters of VHH (final concentration: 1 μM) in triplicate and incubated for 30 minutes. Thereafter, six microliters of a mixture of forskolin and mouse CCL1 (R&D Systems, 845-TC) was added at a final concentration corresponding to its EC80 value. The plates were then incubated for 30 min at room temperature. After addition of the lysis buffer and 1 hour incubation, fluorescence ratios were measured with the HTRF kit (Cisbio, 62AM9PE) according to the manufacturer's specification.

At 1 μM, VHH-01 inhibited CCL1 action on cAMP levels, whereas VHH-06 did not alter cAMP levels over the control (PBS). These data indicate that VHH-01 is a blocking binder of CCR8, while VHH-06 is a non-blocking binder.

### Ca²⁺ Release Assay

The potential of VHH-01 to functionally inhibit mouse CCL1 signalling on CHO-K1 cells displaying mCCR8 was further evaluated in Ca²⁺ release experiments.

Recombinant cells (CHO-K1 mt-aequorin stably expressing mouse CCR8) were grown 18 hours in media without antibiotics and detached gently by flushing with PBSEDTA (5 mM EDTA), recovered by centrifugation and resuspended in assay buffer (DM EM/HAM's F12 with HEPES+0.1% BSA protease free). Cells were then incubated at room temperature for at least 4 hours with Coelenterazine h (Molecular Probes). Thirty minutes after the first injection of 100 μl of a mixture e of cells and VHHs (final concentration: 1 μM), 100 ul of mouse CCL1 (R&D Systems, 845-TC) was added at a final concentration corresponding to its EC80 value and injected into the mixture. The resulting spectral emission was recorded using a Functional Drug Screening System 6000 (FDSS 6000, Hamamatsu).

VHH-01 indeed led to a strong inhibition of Ca²⁺ release by 94%, confirming that VHH-01 is a blocking binder of CCR8.

### Example 3. Generation of CCR8 VHH-Fc Fusions

### Synthesis and Purification of CCR8 VHH-Fc Fusions

VHH-Fc-14 was generated by combining anti-CCR8 VHHs to the mouse IgG2a Fc domain, separated by flexible GlySer linkers (10GS). VHH-Fc-14 contains two VHH-01 binders in addition to two VHH-06 binders. The construct was cloned in a pcDNA3.4 mammalian expression vector, in frame with the mouse Ig heavy chain V region 102 signal peptide to direct the expressed recombinant proteins to the extracellular environment. DNA synthesis and cloning, cell transfection, protein production in Expi293F cells and protein A purification were done by Genscript (GenScript Biotech B.V., Leiden, Netherlands).

### Confirmation of CCR8 Binding by CCR8 VHH-Fc Fusions

The multivalent VHH-Fc fusion VHH-Fc-14 was evaluated for its ability to bind to mouse CCR8 endogenously expressed on BW5147 cells by means of flow cytometry experiments. Cells were incubated with different concentrations of the multivalent VHH-Fc fusion for 30 minutes at 4° C., followed by two washes with FACS buffer, followed by 30 minutes incubation at 4° C. with AF488 goat anti-mouse IgG (Life Technologies, A11029) or AF488 donkey anti-rat IgG (Life Technologies, A21208), followed by two washing steps. Dead cells were stained using TOPRO3 (Thermo Fisher Scientific, T3605). The binding of VHH-Fc-14 has a pEC50 value of 9.14±0.39 M (n=6) (mean±standard deviation).

### Functional Inhibition by CCR8 VHH-Fc Fusions

### Apoptosis Assay

VHH-Fc-14 was tested in an apoptosis assay for its ability to functionally inhibit the action of the agonistic ligand CCL1.

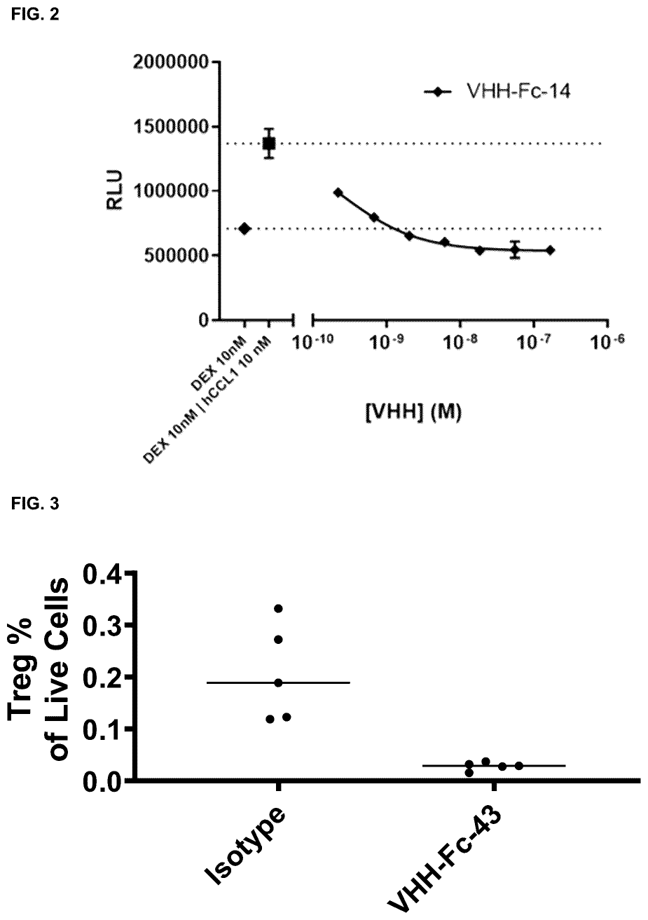

Dexamethasone induces cell death in mouse lymphoma BW5147 cells that endogenously express CCR8. The dexamethasone-induced cell death can be reversed by addition of the antagonist ligand CCL1 (Van Snick et al., 1996, Journal of immunology, 157, 2570-2576; Louahed et al., 2003, European Journal of Immunology, 33, 494-501; Spinetti et al., 2003, Journal of Leukocyte Biology, 73, 201-207; Denis et al., 2012, PLOS One, 7, e34199). 50 μl of cells (seeded at 2.75×10⁴ cells/ml in Iscove-Dulbecco's medium+10% FBS, 50 μM 2-ME, 1.25 mM I-glutamine) were incubated with 30 μl of serial dilutions of the VHH-Fc fusion and incubated for 30 minutes at 37° C. Next, a 20 μl mixture of dexamethasone (Sigma-Aldrich, D4902) and human CCL1 (Biolegend, 582706) was added to a final concentration of 10 nM each. After 48 hours incubation at 37° C., cell viability was quantified using the ATPlite 1-step lit according to the manufacturer's instructions (Perkin Elmer, 6016736). These results of this assessment are depicted in FIG. 2.

The VHH-Fc fusion VHH-Fc-14 provides strong functional inhibition in the assay with a pIC50 value of 9.29±0.22 M (n=9) (mean±standard deviation).

### cAMP Assay

VHH-Fc-14 was tested in the cAMP assay as described in example 2. VHH-Fc-14 provides for a 100% inhibition of the cAMP signal at a concentration of 50 nM and higher, with a pIC50 value of 8.54 M, again confirming that it is a blocking CCR8 binder.

### Example 4. CCR8 VHH-Fc Fusions Affect Intestinal Treg Levels

In order to study the effects of cytotoxic CCR8 binders on intratumoural and other Treg levels, VHH-Fc-14 was modified to obtain VHH-Fc fusions with increased and abolished ADCC activity. Increased ADCC activity was obtained through a-fucosylation of VHH-Fc-14 (VHH-Fc-43). Alternatively, ADCC activity was abolished in VHH-Fc-14 through insertion of the LALAPG Fc mutations (VHH-Fc-41) (Lo et al., 2017, Journal of Biological Chemistry, 292, 3900-3908). Constructs were cloned in mammalian expression vector pQMCF vector in frame with a secretory signal peptide and transfected to CHOEBNALT85 1E9 cells, followed by expression, protein A and gel filtration chromatography (Icosagen Cell Factory, Tartu, Estonia). Versions with α,-fucosylated N-glycans in the CH2 domain of the Fc moiety were obtained from expressions in a CHOEBNALT85 cell line that carries GlymaxX technology (ProBioGen AG, Berlin, Germany) (Icosagen Cell Factory, Tartu, Estonia).Proteins were 0.22 mm sterile filtrated. Protein concentration was determined by measurement of absorbance at 280 nm and purity was determined by SDS-PAGE and size exclusion chromatography. Endotoxin levels were assessed by LAL test (Charles-River Endochrome). The control, mlgG2a isotype, was purchased from BioXCell. VHH-Fc-41 (pEC50 value of 9.33 M (n=1)) and VHH-Fc-43 (pEC50 value of 9.23±0.17 M (n=2)) bind comparably to CCR8 on BW5147 cells. In addition, both VHH-Fc-41 (pIC50 value of 9.51±0.02 M (n=2)) and VHH-Fc-43 (pIC50 value of 9.39±0.11 M (n=4)) (mean±standard deviation) potently inhibit the action of CCL1 in the BW147 apoptosis assay. All values are show as mean±standard deviation.

To test the effects of these blocking CCR8 VHH-Fc fusions with and without ADCC activity, 3×10⁶ cells LLC-OVA cells (200 μl) were subcutaneously injected in female C57BL/6 mice (6-12 weeks). At day 4, mice were treated with 200 μg of anti-CCR8 VHH-Fc (VHH-Fc-41 or VHH-Fc-43) or mouse IgG2a (control) once weekly (i.e. day 4, 11) (n_{mice/group}=5)

At day 16 mice were sacrificed and tumour, blood and intestines were harvested from each mouse.

Tumour single cell suspensions were obtained by cutting the tissues in small pieces, followed by treatment with 10 U ml-1 collagenase I, 400 U ml-1 collagenase IV and 30 U ml-1 DNasel (Worthington) for 25 minutes at 37° C. The tissues were subsequently squashed and filtered (70 μm). The obtained cell suspensions were removed of red blood cells using erythrocyte lysis buffer (155 mM NH4Cl, 10 mM KHCO3, 500 mM EDTA), followed by neutralization with RPMI. Blood was depleted of red blood cells through repeated rounds of incubation for 5 minutes in erythrocyte lysis buffer until only leukocytes remained.

Intestinal single cell suspensions were prepared as previously described (C. C. Bain, A. Mcl. Mowat, CD200 receptor and macrophage function in the intestine, Immunobiology 217, 643-651 (2012)). After erythrocyte lysis, the obtained single cell suspensions were resuspended in FACS buffer (PBS enriched with 2% FCS and 2 mM EDTA) and counted. All single cell suspensions were pre-incubated with rat anti-mouse CD16/CD32 (2.4G2; BD Biosciences) or anti-human Fc block reagent (Miltenyi) for 15 minutes prior to staining. After washing, the samples were stained with fixable viability dye eFluor506 (eBioscience) (1:200) for 30 minutes at 4° C. and in the dark. Subsequently, the samples were washed and stained for 30 minutes at 4° C. and in the dark. The intracellular staining of cytokines/chemokines and transcription factors was done according to the manufacturers protocol (Cat No. 554715; BD Biosciences) and (Cat No. 00-5523; Invitrogen), respectively. FACS data were acquired using the BD FACSCantoll (BD Biosciences) and analyzed using FlowJo (TreeStar, Inc.).

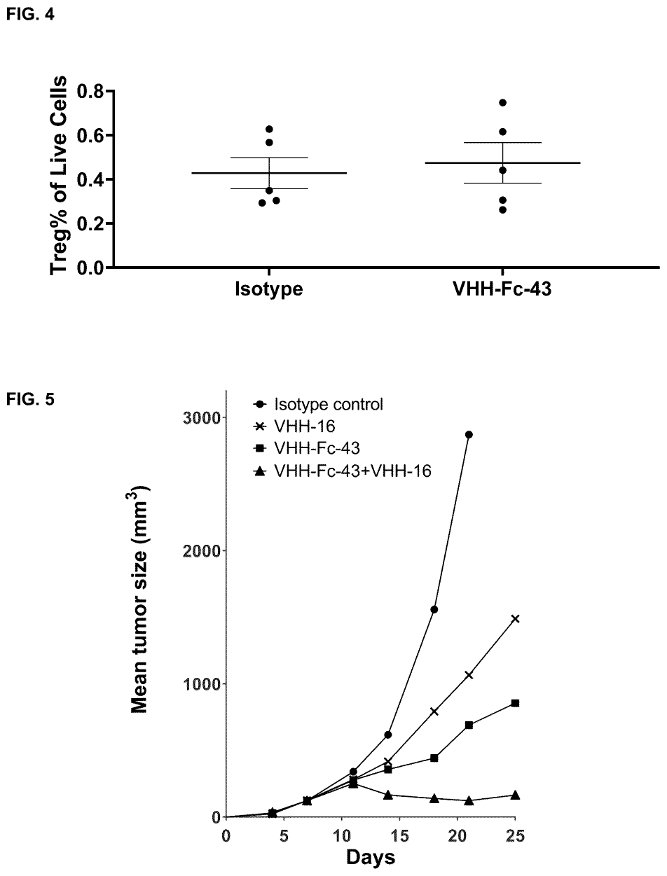

As is shown in FIG. 3, Tregs are depleted in the tumour by VHH-Fc-43, which is a CCR8 blocking Fc fusion with ADCC activity, while no intratumoural Treg depletion is observed for VHH-Fc-41, which lacks ADCC activity. No depletion of circulating Tregs was observed for either construct (FIG. 4).

### Example 5: Generation of LTBR Agonistic Single Domain Antibody Moieties

### Immunizations

VHHs were generated through immunization of llamas and alpacas with recombinant protein, essentially as described elsewhere (Pardon et al., 2014) (Henry and MacKenzie, 2018). Briefly, animals were immunized six times at one week intervals with 50 [symbol] g of recombinant mouse LTBR-mouse IgG2A Fc chimera protein (R&D Systems, cat. #1008-LR) after which blood samples were taken.

### Phage Display Library Preparation

Phage display libraries derived from peripheral blood mononuclear cells (PBLCs) were prepared and used as described elsewhere (Pardon et al., 2014; Henry and MacKenzie, 2018). The VHH fragments were inserted into a M13 phagemid vector containing MYC and His6 tags. The libraries were rescued by infecting exponentially-growing Escherichia coli TG1 [(F′ traD36 proAB laclqZ ΔM15) supE thi-1 Δ(lac-proAB) Δ(mcrB-hsdSM)5(rK−mK−)] cells followed by surinfection with VCSM13 helper phage. The mouse LTBR immunized phage libraries were subjected to two consecutive selection rounds on mouse LTBR—mouse IgG2A Fc chimera protein (R&D Systems, cat. #1008-LR), in the presence of a 50-fold excess of total mouse IgG to eliminate Fc-binding VHHs. Individual colonies were grown in 96-deep-well plates from E. coli TG1 cells that were infected with the eluted phages from the different selection rounds. Monoclonal VHHs were expressed essentially as described before (Pardon et al., 2014). The crude periplasmic extracts containing the VHHs were prepared by freezing the bacterial pellets overnight followed by resuspension in PBS and centrifugation to remove cell debris.

### Screening of LTBR Selection Outputs

VHHs clones from the immunization and selection campaign were screened as crude periplasmic extracts by means of binding ELISA to mouse LTBR compared to uncoated controls. Binding was confirmed by means of biolayer interferometry

### ELISA

1 μg/ml of mLTBR-mFc (R&D Systems, cat. #1008-LR) diluted in PBS at pH 7.4 was coated on 96-well microtiter plates followed by blocking with 4% dry skimmed milk in PBS (Marvel). Next, 1:5 dilutions of crude periplasmic extracts from monoclonal VHH clones were added, followed by detection with 1:1000 anti-c-myc antibody 9E10 (Merck, cat. #11667203001) and anti-mouse IgG-HRP (Jackson Immuno Research, cat. #715-035-150) at a 1:5000 dilution, both in 1% dry skimmed milk in PBS. In between applications, plates were washed with PBS supplemented with Tween 0.05% pH7.4. Reaction development was done using 100 μl of HRP substrate TMB (Thermo Fisher, cat. #00-4201-56). The reaction was stopped by addition of 1001110.5 M H₂SO₄ (Fisher Scientific, cat. #J/8430/15) and read out on a plate reader at OD₄₅₀. Clone P002MP07G04 had an OD₄₅₀ binding signal of 4.458 to mLTBR-mFc versus 0.042 on the uncoated control.

### Biolayer Interferometry (BLI)

Bio-Layer Interferometry (BLI) is a label-free technology for measuring biomolecular interactions that analyzes the interference pattern of white light reflected from two surfaces, a layer of immobilized protein on the biosensor tip and an internal reference layer. Any change in the number of molecules bound to the biosensor tip causes a shift in the interference pattern that can be measured in real-time. The binding between a ligand immobilized on the biosensor tip surface and an analyte in solution produces an increase in optical thickness at the biosensor tip, which results in a wavelength shift, which is a direct measure of the change in thickness of the biological layer. Kinetic binding parameters off-rate (k_{off}) and dissociation constant (K_{D}) were determined on an Octet RED96e machine (ForteBio) according to the manufacturer's procedures and analyzed using the Data Analysis 9.0 software (ForteBio). Mouse LTBR-Fc (R&D Systems, cat. #1008-LR) captured on anti-murine IgG Fc capture (ForteBio, cat. #18-5088) tips was dipped in ⅕ diluted periplasmic extract of clone P002MP07G04, resulting in a k_{off} value of 1.8×10⁻⁰² S⁻¹.

### Reporter Assay

Clone P002MP07G04 was displayed in multimeric fashion on top of monoclonal phage particles, and screened in the reporter assay to evaluate its agonistic potential in comparison to irrelevant controls. Two different formats of monoclonal phages were thus evaluated: (i) VCSM13-rescued phages that display a range (one to five) of VHH fragments per phage particle and (ii) Hyperphage-rescued phages (Progen, cat. #PRHYPE-XS) that display five VHH fragments per phage particle. Clone P002MP07G04 thus yielded a reporter assay signal ratio compared to an irrelevant control of respectively 4.7 and 3.2, suggesting that a multivalent display of P002MP07G04 is able to activate mouse LTBR.

### Production, Purification and In Vitro Characterization of Monovalent LTBR VHHs

Synthetic DNA fragments encoding VHHs were ordered and subcloned into an E. coli expression vector under control of an IPTG-inducible lac promoter, in frame with N-terminal PeIB signal peptide (which directs the recombinant proteins to the periplasmic compartment) and C-terminal FLAG3 and HIS6 tags. Electrocompetent E. coli TG1 cells were transformed and the resulting clones were sequence verified. VHH proteins were purified from these clones by means of IMAC chromatography followed by desalting according to well established procedures (Pardon et al., 2014).

A binding KD of 55 nM for purified monovalent P002MP07G04 to mouse LTBR-Fc (R&D Systems, cat. #1008-LR) captured on anti-murine IgG Fc capture (ForteBio, cat. #18-5088) tips was determined by means of BLI.

100 nM of purified monovalent P002MP07G04 was cross-linked through its C-terminal HIS6 tag by an anti-His tag mAb (Genscript, cat. #A00186-100) at a 2:1 molar ratio. This dimeric display of P002MP07G04 imparted LTBR agonism in the reporter assay with an NFκB signal to background ratio of 6.8. In contrast, non-cross-linked monovalent P002MP07G04 was not active at 100 nM in the reporter assay.

### Production, Purification and In Vitro Characterization of Multivalent LTBR VHHs

VHH-16, a tetravalent VHH combining three P002MP07G04 building blocks and one anti-serum albumin building block SA26h5 (WO/2019/016237), separated by 20GS flexible GlySer linkers, was generated essentially as described before (Maussang et al., 2013; De Tavernier et al., 2016). The multivalent construct was cloned and sequence-verified in a Pichia pastoris expression vector under control of an AOX1 methanol-inducible promoter, in frame with an N-terminal Saccharomyces cerevisiae alpha mating factor signal peptide that directs the expressed recombinant proteins to the extracellular environment. Transformation and expression in Pichia pastoris and purification by means of protein A purification were done essentially as described before (Lin-Cereghino et al., 2005; Schotte et al., 2016). When tested in the reporter assay, VHH-16 activated mouse LTBR with a mean (±standard deviation) pEC50 value of 9.35±0.03 (n=3).

### Example 6: Effects of a Treg Depletor in Combination with and LTBR Agonist on Tumour Growth in an MC38 Syngeneic Mouse Model

The mouse MC38 tumour model was used to test the efficacy of the mono- and combination therapy of anti-CCR8, using VHH-Fc-43, and an LTBR agonist, using VHH-16.

At day 0, 5×10⁵ MC38 cells (0.1 ml cell suspension) was injected subcutaneously into the right flank of 8 week old female C57BL/6J mice. At day 7, animals reached an average tumor size of approximately 125 mm³ and were sorted into 4 groups of 10 each. Mice were injected biweekly for 3 weeks with 200 μg mouse IgG2a, 200 μg P00500043, 40 μg VHH-16, or a combination of 200 μg VHH-FC-43+40 μg VHH-16. Weights and tumor burdens were measured biweekly for the duration of the 3 week trial. Tumours were measured with a caliper in two dimensions to monitor growth, and mice were sacrificed when their tumours exceeded the ethical endpoint of 2000 mm³.

Tumor size, in mm³, was calculated from:

Tumor Volume=(w²×l)×0.52
where w=width and l=length, in mm, of the tumor

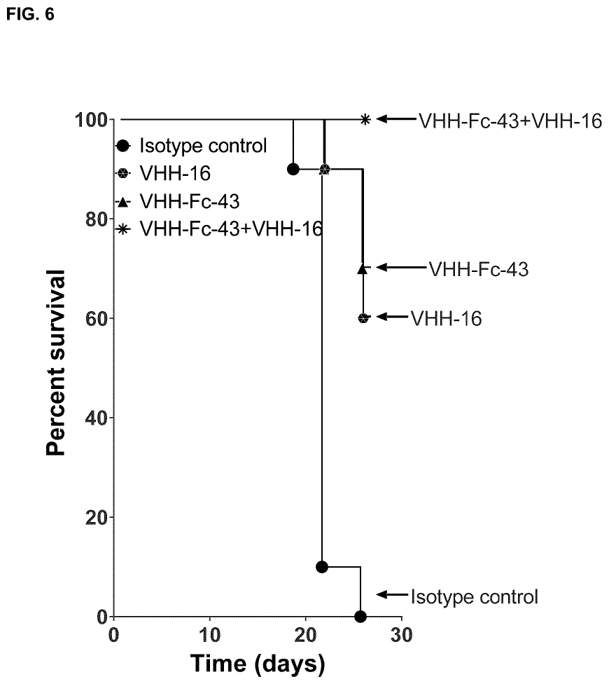

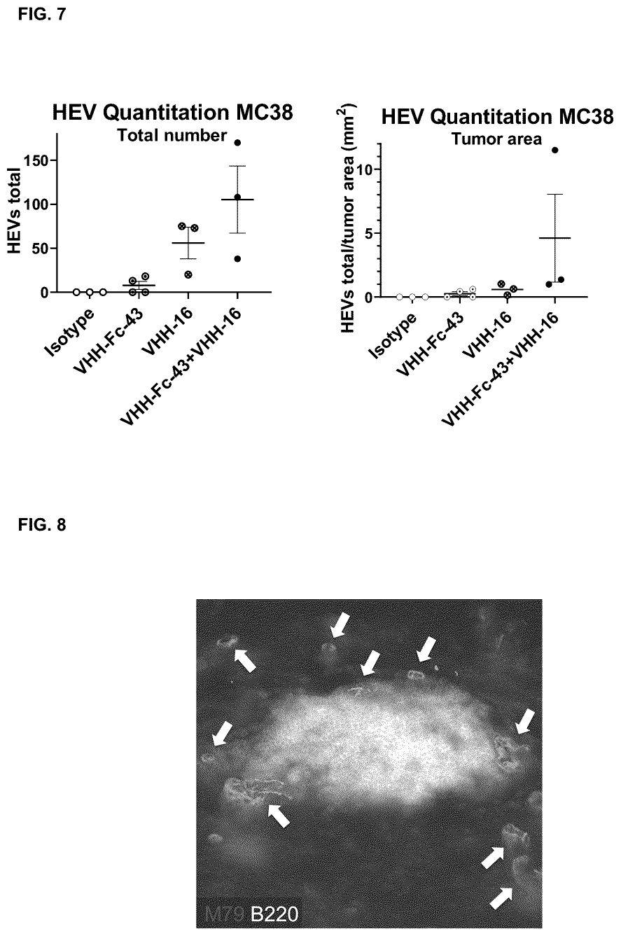

The mean tumor size for the four cohorts are depicted in FIG. 5 commencing from day 0 to day 25. While both monotherapies are effective at controlling tumour growth from day 14-25 versus isotype controls, the combination anti-CCR8 and LTBR agonist treatment additionally produces synergism in reducing tumour burden starting at day 14 and commencing to end stage at day 25 versus both monotherapies. This is also reflected in the Kaplan-Meier survival curves that show that while all isotype treated animals ( 10/10) reached the ethical endpoint of 2000 mm³ by day 25, only 3/10 VHH-FC-43 and 4/10 VHH-16 monotherapy treated animals reached endstage. Moreover, no mice ( 0/10) treated with combination VHH-FC-43+VH H-16 therapy reached endstage (FIG. 6). Two-way ANOVA with mixed effects model comparing the various treatment arms indicates that there is statistically significant difference between both mono- and combination therapy versus isotype controls from day 14 to day 21 (when 9/10 mice are sacrificed due to high tumour burden), and that the combination therapy is statistically superior to VHH-16 from day 14-25, and to VHH-FC-43 at day 14. The log rank test was performed using the survival data and showed that survival was increased between all treated arms and isotype controls, and also for VHH-16 monotherapy versus combination therapy (p-value=0.0297). There is a trend towards increased survival for combination therapy vs VHH-FC-43 (p-value=0.0676). FIG. 7 shows quantitation of the numbers of high endothelial venules (HEVs) found in isotype and treated tumours for all cohorts along with the number of HEVs/tumour area. Immunofluorescence staining was performed on tumours stained with the peripheral node addressin antibody, AF488 anti-MECA79 (M79). When a putative HEV was identified, blood vessel staining was assessed using AF568 anti-CD31. If an HEV is present, there is discontinuous MECA79 signal on the luminal side of the CD31 positive blood vessel, which stains continuously. Two sections from each tumor were manually counted and averaged from 3-4 treated mice for each condition, and tumor area was calculated from the area of DAPI-positive nuclei using the Zen Blue software program. The results show an increased induction of HEVs in the combination treated tumors versus each monotherapy, and the localization of HEVs shifts from the tumour periphery in VHH-16 monotherapy treated mice to deep within the tumour in combination treated animals (data not shown). In addition, “mature” appearing tertiary lymphoid structures (TLSs), consisting of numerous MECA-79 positive HEVs (arrows) surrounding an organized structure consisting of copious B220 positive B cells, are found in 4/6 combination treated tumours (FIG. 8) In addition, some HEVs deep within the combination treated tumours were surrounded by numerous individual B cells. Collectively, the reduction in tumour burden, trend toward increased survival, and increased HEV and TLS induction in combination treated animals shows the synergistic activity of LTBR agonism and Treg depletion therapy.

### Example 7: Effects of a Treg Depleting Anti-CTLA4 in Combination with an LTBR Agonist on Tumour Growth in an MC38 Syngeneic Mouse Model

The mouse MC38 tumour model was used to test the efficacy of the mono- and combination therapy of anti-CTLA4, having Treg depletion activity, and an LTBR agonist, using VHH-16. The anti-CTLA4 antibody used in these experiments is based on the the previously described anti-mCTLA4 9D9 antibody, but wherein the murine IgG2b has been replaced with murine IgG2a constant region. Murine IgG2a was chosen because it provides for stronger ADCC activity in mice (Selby M J. et al., 2013. Anti-CTLA-4 antibodies of IgG2a isotype enhance antitumor activity through reduction of intratumoral regulatory T cells. Cancer Immunol Res. 1(1):32-42).

At day 0, 5×10⁵ MC38 cells (100 μL) was injected subcutaneously in female C57BL/6J mice (7-9 weeks). At day 7, animals reached an average tumor size of approximately 116 mm³ and were sorted into 4 groups of 10 each, i.e. mouse IgG2a (control), anti-CTLA4 monotherapy, CHH-16 monotherapy and combination of anti-CTLA4+VH H-16. Mice were intraperitoneally injected biweekly for 3 weeks with 200 μg mouse IgG2a (control) and 40 μg VHH-16, starting on day 7. Treatment with 200 μg of anti-CTLA4 started on day 10 and mice were dosed once weekly for 3 weeks. Weights and tumor burdens were measured biweekly for the duration of the 3 week trial. Tumours were measured with a caliper in two dimensions to monitor growth. Tumor size, in mm³, was calculated from:

Tumor Volume=(w²×l)×0.52
where w=width and l=length, in mm, of the tumor

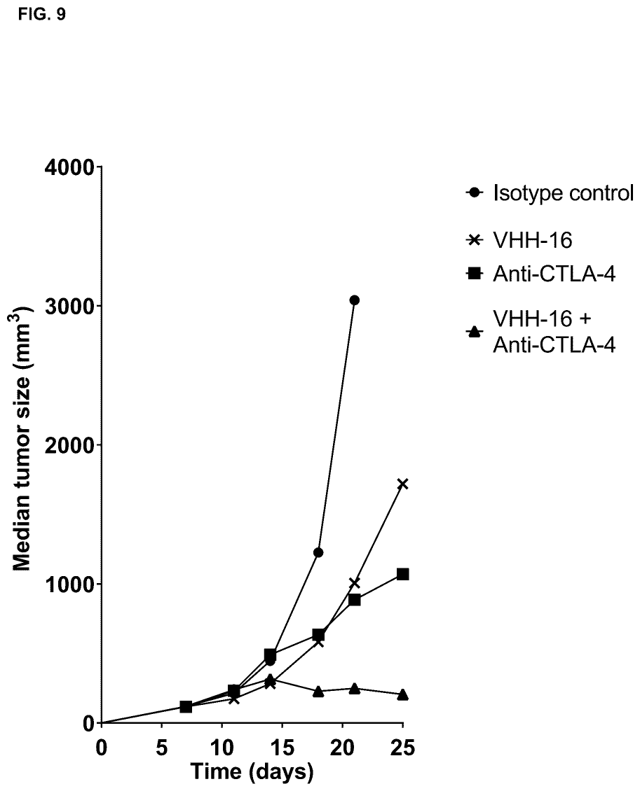

The median tumor size (in mm³) for the four cohorts are depicted in FIG. 9 commencing from day to day 25. The cohorts treated with anti-CTLA4 and VHH-16 as monotherapy showed from day 18 a lower tumour size in comparison with the isotype control. Additionally, the combination of anti-CTLA4 Treg depletion and LTBR agonist treatment produced synergism in reducing tumour burden and even leading to tumour stasis or regression in a majority of the mice in this treatment group.

1. A combination comprising:
a Lymphotoxin Beta Receptor (LTBR) agonist; and
a regulatory T cell (Treg) depletor.

2. The combination of claim 1, wherein the Treg depletor binds to a cell surface marker of a Treg and has cytotoxic activity.

3. The combination according to of claim 2, wherein the cell surface marker of the Treg is selected from the group consisting of CCR8, CCR4, CTLA4, CD25, TIGIT, OX40, ICOS, CD38, GITR, 4-1BB, NRP1, and LAG-3.

4. The combination of claim 2, wherein the cell surface marker of the Treg is CCR8 or CTLA4.

5. The combination of claim 2, wherein the cytotoxic activity of the Treg depletor is caused by the presence of a cytotoxic moiety that
induces antibody-dependent cellular cytotoxicity (ADCC),
induces complement-dependent cytotoxicity (CDC),
induces antibody-dependent cellular phagocytosis (ADCP),
binds to and activates cytotoxic T-cells or T helper cells, or
comprises a cytotoxic payload.

6. The combination of claim 5, wherein the cytotoxic moiety comprises a fragment crystallisable (Fc) region moiety, in particular an Fc region moiety that has been engineered to increase ADCC, CDC, and/or ADCP activity.

7. The combination of claim 1, wherein the Treg depletor is a CCR8 binding antibody having ADCC, CDC or ADCP activity.

8. A composition comprising the combination of claim 1.

9. A The combination of claim 1, wherein the LTBR agonist is an LTBR agonistic moiety and the Treg depletor is a Treg depleting moiety, wherein the LTBR agnoistic moiety and the Treg depleting moiety are comprised in a bispecific molecule having cytotoxic activity.

10. (canceled)

11. A method for the treatment of a cancer, the method comprising administering to a subject suffering from the cancer the combination of claim 1.

12. The the method according to claim 11, wherein the cancer is selected from the group consisting of a breast cancer, uterine corpus cancer, lung cancer, stomach cancer, head and neck squamous cell carcinoma, skin cancer, colorectal cancer, and kidney cancer.

13. A method for the treatment of a cancer, the method comprising:
administering to a subject suffering from the cancer an LTBR agonist, and
treating the subject with Treg depletion therapy.

14. The LTBR agonist for use the method according to claim 13,
wherein the LTBR agonist is an LTBR agonistic antibody; and
wherein the Treg depletion therapy comprises the administration of a CCR8 binding antibody having ADCC, CDC and/or ADCP activity.

15. (canceled)

16. The combination of claim 6, wherein the engineering to increase ADCC, CDC, and/or ADCP activity is afucosylation or an ADCC, CDC and/or ADCP-increasing mutation
